(19) United States
(12) Patent Application Publication
(10) Pub. No.: US 2006/0122863 A1
(43) Pub. Date: Jun. 8, 2006

(54) Patient management network

(75) Inventors: Janell M. Gottesman, St. Louis Park, MN (US); H. T. Markowitz, Roseville, MN (US); James E. Willenbring, St. Paul, MN (US); Christopher M. Manrodt, White Bear Lake, MN (US); John P. Vandanacker, Greenfiled, MN (US); John E. Burnes, Andover, MN (US); John C. Rueter, Woodbury, MN (US); Sarah A. Audet, Shoreview, MN (US); James K. Carney, Eden Prairie, MN (US); Gerard J. Hill, Champlin, MN (US)

Correspondence Address: MEDTRONIC, INC., 710 MEDTRONIC PARK, MINNEAPOLIS, MN, 55432-9924, US

(73) Assignee: Medtronic, Inc.

(21) Appl. No.: 11/038,814

(22) Filed: Jan. 20, 2005

Related U.S. Application Data
(60) Provisional application No. 60/632,642, filed on Dec. 2, 2004.

(51) Int. Cl. G06Q 10/00; A61K 9/22

(52) U.S. Cl. 705/2; 604/891.1

(57) ABSTRACT

An intelligent patient management system collects data from a variety of sources, processes that information, and provides relevant information to an appropriate caregiver in context at the proper time. The system has multiple information sources available from which to gather additional information based upon conclusions drawn or issues identified from the patient data.

### BACKGROUND OF THE INVENTION

1. Field of the Invention

The present invention relates to management of patient data and more specifically to collection, management and presentation of patient medical data.

2. Description of the Related Art

In order for a caregiver to provide proper medical evaluation or assistance, that caregiver must have knowledge in two categories. First, the caregiver must have the proper education, experience and access to current medical information in order to be competent. Second, that caregiver needs to have information relating to the patient and the patient's immediate concerns.

In some instances, the patient's immediate concern may be so specific that this second category of information may be minimal. For example, a caregiver may witness a person choking on an object. Without knowing anymore about the “patient”, that caregiver can dislodge the object, restore breathing and in effect restore this patient's health. Typically, this patient would not need any subsequent follow-up for this issue.

In other, seemingly simple, medical situations additional information is required to safely treat a patient. For example, a parent may bring a child to a caregiver complaining of ear pain. An infection is discovered and an oral or topical antibiotic seems an obvious treatment. However, the caregiver will ask the parent if the child has any known allergies. If the child is allergic to the likely antibiotic, this piece of patient information becomes critical.

It is thus readily apparent why patient medical records are important to the caregiver and to the patient. That record, if available, accurate, and complete, can provide the caregiver with the relevant information to avert, for example, prescribing a drug the patient is allergic to if the patient cannot provide such information directly or reliably. As indicated, this information is only useful when it is available to the caregiver. Many clinics still rely on paper files and this limits access to a single location. Even many current electronic records may limit or preclude access to other caregivers or other institutions for any number of reasons.

Now consider a patient having a drug allergy and is incapable of providing information (e.g., too young, unconscious, etc.) and is being seen by a caregiver that does not have access to that patient's medical record. One option for that patient is to carry with them information relating to this allergy. Often, this may be in the form of a “Medic Alert” bracelet or necklace. As these are generally known sources of information, emergency caregivers will often check for their presence. Thought of broadly, the patient is providing a source of data to any caregiver and is providing at least a small medical record with potentially relevant information in a timely manner. Of course, the practical reality is that only small amounts of very specific information may be catalogued and presented in this manner. Thus, in addressing the above overall concerns with patient records, the data is available and presumably accurate, but is in no sense complete.

These examples have illustrated acute care situations for a given patient. A large segment of health care is devoted to preventative medicine and to treatment of long term conditions. For example, a patient having hypertension may be seen many times while possible pharmaceutical options are explored. Once an appropriate medication and dosage has been established, the caregiver will continue to regularly see the patient to evaluate efficacy and make any required modifications.

This course of treatment is based on blood pressure measurements made over time during each office visit and recorded in the patient's medical record. Thus, the caregiver can review this record and use such knowledge in making subsequent medical decisions. For example, during an office visit a hypertensive patient has elevated blood pressure measurements. Taken in isolation, this data would suggest that the caregiver increase a dosage or otherwise modify the patient's medication. However, upon reviewing the medical record, the caregiver determines that this drug regime has successfully treated the hypertension for a long period of time. Therefore, the caregiver would consider other reasons for a spurious or abnormally high measurement; that is, the caregiver would have reason to assume that the current measurements could be abnormal rather than necessarily indicative of the patient's general state.

This rather simple example illustrates another aspect of the above noted concerns with medical records; namely, that they are complete. Any given blood pressure measurement (i.e., any given data point) might not provide a complete assessment of the patient's state. Furthermore, though such an individual measurement is probably correct, when relied upon in isolation it may, in fact, not provide an accurate assessment. Therefore, a collection of data over time is more complete and provides a more accurate assessment.

Continuing with this example, regularly scheduled office visits to monitor blood pressure still might not provide a complete and therefore accurate assessment of the patient's hypertension. That is, each in-office visit is still simply an isolated measurement at one point in time. Furthermore, the measurement may very well affect the outcome. Patient stress associated with medical evaluation may artificially increase blood pressure.

Where this is a concern, patients may obtain the means to monitor their own blood pressure and record measurements more frequently and without the stress created by a medical evaluation. This data can then be presented to the caregiver and included in the medical record. This requires consideration of the patient's ability to make and record such measurements as well as the accuracy and calibration of the patient's equipment relative to that of the caregiver.

A more accurate and more complete system for managing patient long-term treatment is the automated collection and recording of patient data. For example, the Medtronic Reveal® is a subcutaneous implantable loop recorder that senses electrical activity correlated with cardiac rhythm. Data is automatically collected over time and recorded into the device memory. Subsequently, this data is retrieved by a caregiver and evaluated. This data provides a more complete and more accurate data set than is individually collected in office visits. The Medtronic Reveal® is used, for example, to monitor cardiac activity during syncope. The particular sensed cardiac data could easily be acquired during an in-office visit; however, the odds of having a syncopic episode during such an evaluation are extremely low. Thus, the accuracy of the data collected in-office is high, but it is incomplete as compared to that collected by the implantable loop recorder.

Various other implantable medical devices (IMD) sense and record data. For example, implantable pacemakers and defibrillators sense cardiac activity in order to determine and provide therapy. This data along with device specific parameters are often recorded in memory and are subsequently retrievable by a caregiver. Generally, this has required the patient to attend an in-office visit. The caregiver utilizes a programmer, such as the Medtronic Model 2090™ Programmer, to retrieve the data. A programming head, coupled to the programmer is placed in close proximity to the IMD and data is collected via telemetry.

The collected data may provide a broad range of usefulness. For example, the data may include very device specific parameters such as remaining battery life or lead integrity. This data is extremely relevant to the caregiver responsible for the device, such as an electrophysiologist (EP), cardiologist, and/or their staff, but would likely be of little value to other caregivers in many situations. In addition, the data may indicate clinical parameters such as the onset, nature, and parameters of various arrhythmias. This data is likewise extremely relevant to the same group of caregivers responsible for the device; however, such data may also be quite relevant to other caregivers attending to the same patient. For example, the onset of an arrhythmia may be related to a drug being prescribed to treat a separate condition; that this drug is having such side effect would be useful information to the caregiver prescribing that drug.

This introduces a subsequent challenge for medical data usage and management. As indicated, this data must be available, accurate and complete, but the relevance of the data must be apparent to the caregiver. For example, if in the above example a general practitioner (GP) is prescribing the drug that is associated with the arrhythmias, a device output report as formatted and provided to an EP may make little sense to that GP. The general practitioner might not readily recognize the arrhythmia data and its relevance to the care provided. Furthermore, even the opportunity to make such an assessment assumes the general practitioner has that data available or would know to request that data, which is often not the case.

Implantable medical devices have limited memory capacity and their use to collect data as described requires in-office visits. These are both time consuming and limiting for both the caregiver and the patient. Thus, Medtronic has provided the Medtronic CareLink® network. Patients are provided with a home monitor that communicates via telemetry with the implantable device. Data is transferred via the home monitor to a central database. This data is then formatted and made available to the caregiver. This provides numerous advantages to both the patient and the caregiver, including reduced in-office visits, frequent and reliable data collection, and the ability to remotely manage the patient.

Thus, device data that is accurate and complete is made more available via such a network and service. This information can become part of the patient's medical record, either manually or by direct importation into an electronic medical record (EMR).

### BRIEF DESCRIPTION OF THE DRAWINGS

FIG. 1 is a schematic diagram illustrating an intelligent patient management system.

FIGS. 2-4 are schematic diagrams of subsets of the intelligent patient management system of FIG. 1

FIG. 5 is a schematic diagram illustrating a privacy filter.

FIG. 6 is a flowchart illustrating a process for identifying information that is requested by the system.

FIG. 7 is a flowchart illustrating a process for collecting and presenting information in context.

FIG. 8 is a block diagram illustrating a process of gathering data.

FIG. 9 is a flowchart illustrating data processing.

FIG. 10 is a flowchart illustrating a process for providing context.

FIG. 11 is a schematic illustration of a database structure.

FIG. 12 is a flowchart illustrating a process of determining informational needs of a recipient.

FIG. 13 is a block diagram illustrating data sources.

FIG. 14 is a flowchart illustrating a disease management process.

### DETAILED DESCRIPTION

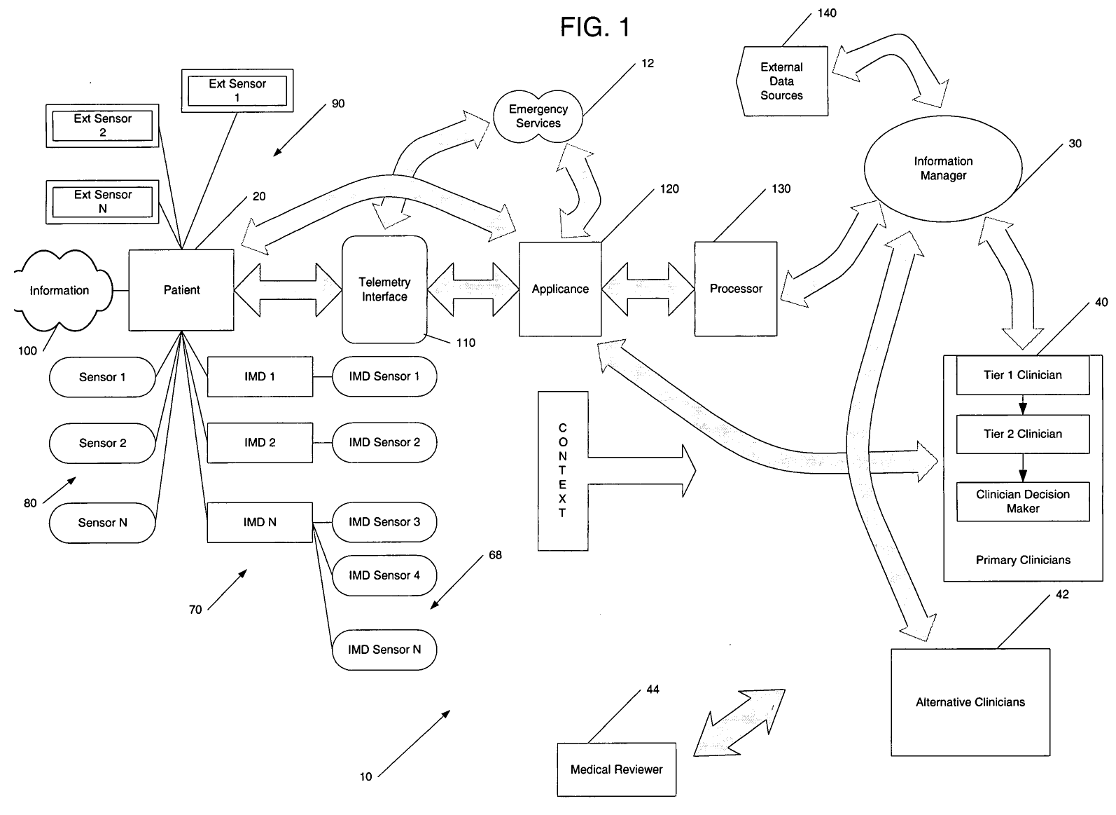

FIG. 1 is a schematic diagram illustrating an intelligent patient management system 10 that collects and provides information, generates context for such information and provides that information in context to an appropriate recipient. System 10 is functionally interactive in assessing collected information, determining appropriate recipients, and identifying the contextual needs of those recipients. If insufficient information is available to provide full context, the system 10 identifies sources for alternative or additional information to provide as much context as possible.

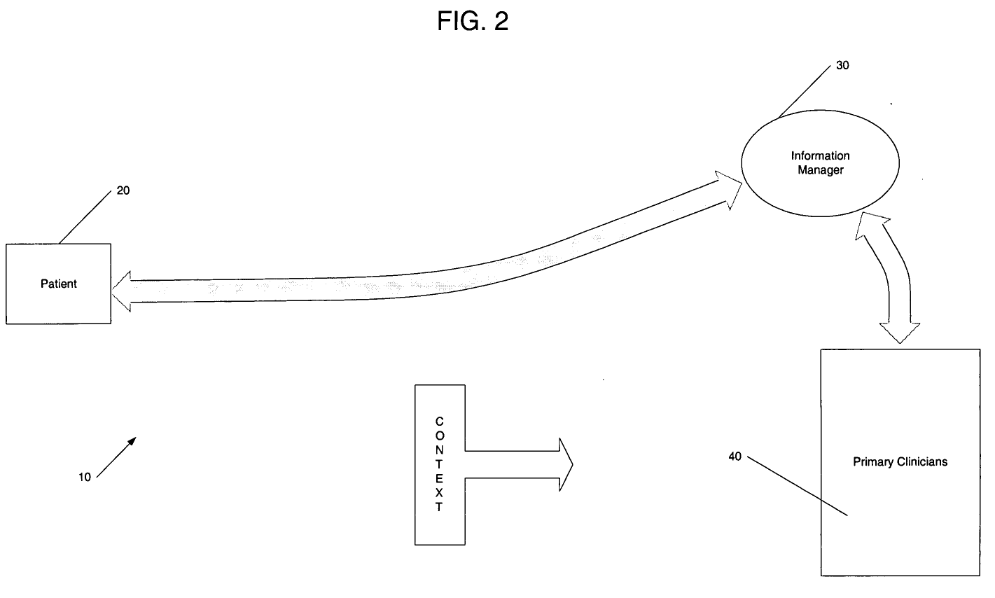

FIG. 2 illustrates three categorical elements of system 10. Specifically, a patient 20, an information manager 30 and the primary clinician 40 are illustrated. The information manager 30 gathers information from and about the patient 20. The information manager 30 evaluates that information and identifies the recipient, illustrated as primary clinician 40.

The term primary clinician is simply meant to indicate the recipient most relevant to the current data or issue being addressed, at least initially. For example, a patient with an implantable cardioverter defibrillator (ICD) would be enrolled on the system and cardiac and device data would be provided to that patient's cardiologist, EP, their staff, or the appropriate caregiver. As will be explained, the system 10 may subsequently determine that the data gathered is also relevant to a different caregiver, e.g., an endocrinologist. In that instance, the endocrinologist would be a secondary or alternative clinician 42 (FIG. 1).

The information manager 30 identifies the recipient, in this case the primary clinician 40. Identification of the recipient may range from a simple assumption that the data will be presented to the enrolling caregiver to an analysis of the data itself to identify other caregivers based on the content, outcome, and conclusions drawn from the analysis of that data by the information manager 30.

When the recipient is identified, the information manager 30 evaluates the data and places that data in an appropriate context for primary clinician 40. The information manager 30 may determine that additional information is required or desired and will attempt to obtain that information to further define the context. This additional information may be obtained from the patient 20 or from other external sources. Once the context is defined, the appropriate and relevant data is provided in context to the primary clinician 40. Based upon the data in context, the primary clinician 40 may take any number of actions including modifying a therapy, maintaining a current therapy, initiating an emergency response, requesting a follow up with the patient (in person or remotely), or requesting additional general or specific information. In addition, remote communication between the primary clinician 40 and the patient 20 may take place, either directly or via the information manager 30. The physician could also be prompted to collect additional specific information which may help the diagnosis and/or better comply with standard clinical practice.

Based upon actions taken by the primary clinician 40, the patient's medical record (either accessible by or maintained by information manager 30) is updated. This may prompt information manager 30 to obtain data from the patient and/or modify the context for subsequent communications. For example, the patient 20 may have an ICD and based upon data presented, the primary clinician 40 prescribes a new drug regiment. This information is made available to the information manager 30, which in turn enables a sensor to monitor for a possible side effect of that drug and/or monitor the efficacy of that drug for it intended purpose. Alternatively, such sensor(s) may be employed to monitor for more general side effects when any new drug is utilized, such as monitoring respiration, which would reveal e.g., a severe allergic reaction.

Assuming that the patient 20 did have a severe allergic reaction to the drug, the information manager 30 would assemble this information and provide that information to emergency services 12 (FIG. 1). Now, as opposed to providing data in a form relevant to e.g., a cardiologist, the information manager 30 is providing the most relevant information in context so that a proper emergency response can be initiated. Relevant information might include the fact that the patient has an ICD, but probably would not include pacing thresholds, PVC's, battery status, mode selections, etc. Rather, information such as the patient's location, the identification of the drug, the nature of the emergency, i.e., an allergic reaction to the drug is preventing respiration, and similar information is presented to facilitate the emergency response. Similarly, when data is presented to alternative caregivers, the appropriate data for that caregiver is selected and/or obtained and presented in a manner relevant to that alternative caregiver.

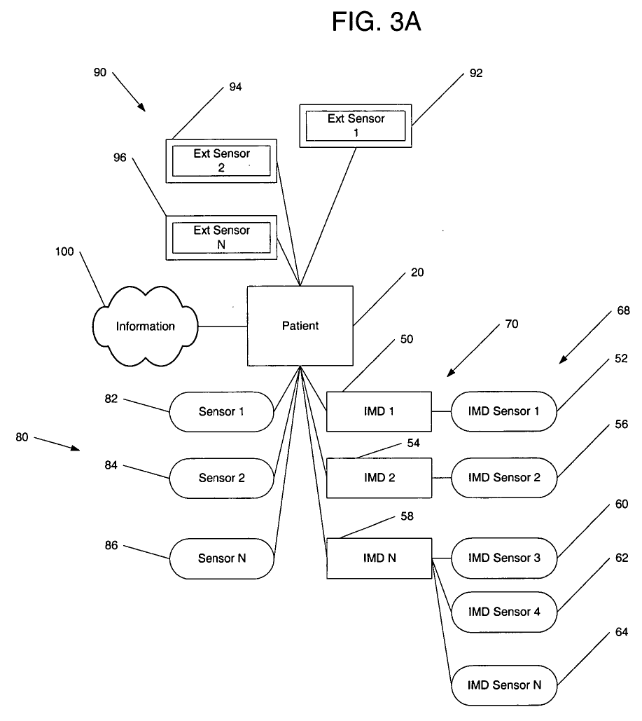

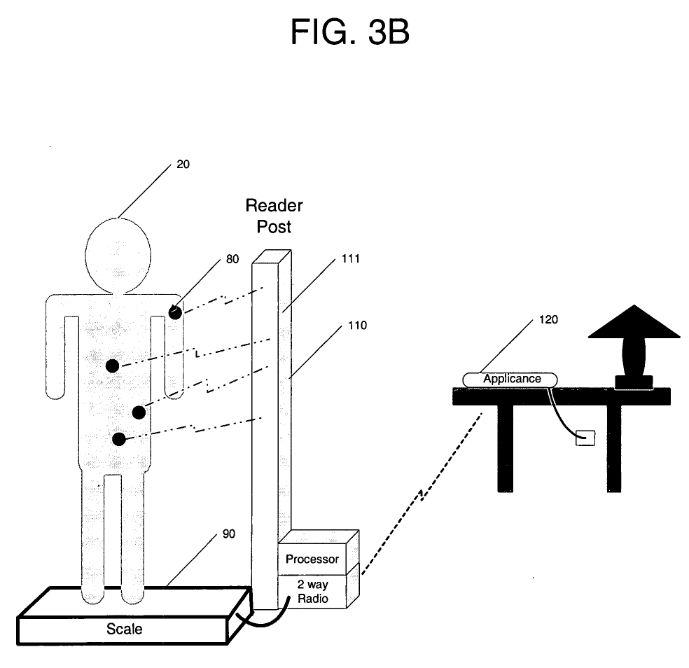

FIG. 3A illustrates a patient subset of system 10 from FIG. 1. The patient 20 may have one or more implantable medical devices (IMD) 50, 54, 58 (collectively 70). Each IMD 70 will have a given function(s) and may deliver therapy. In addition, each IMD 70 may have any number of sensors 52, 56, 60, 62 and 64 (collectively IMD sensors 68) that provide data to the IMD 70 to monitor or control that therapy. The IMD sensors 68 could also be additional sensing platforms that are supplemental to the primary function of the IMD 70. The various IMDs 70 may communicate with each other via telemetry or hardwired connections and thus, one IMD 70 may utilize the sensor data from another IMD 70. The data collected by the sensors 68 is utilized by the system 10 and may be stored within a memory of a given IMD 70 for subsequent retrieval or may be sent to another portion of the system 10 upon collection.

As used herein, the IMD 70 may be any electrical, mechanical, chemical, biological, or genetic device and/or agent or any combination thereof that either provides information to the system 10 and/or responds to direction from the system 10. For example, an ICD is exemplary in that such a device senses and collects information about the patient 20, about its own status, and can be controlled by the system 10 to alter its own programming and affect therapy.

The information provided may be directly from the IMD 70 or from a supplemental sensor or device monitoring the IMD 70. Thus, traditionally non-communicative and/or static devices may be incorporated in the system 10. A stent, in and of itself, is an independent mechanical structure. However, an additional sensor may be implanted to monitor an effect such as blood flow, turbulence, pressure, or an electromagnetic field. These criteria will provide an indication of the presence and/or performance of the stent and that information is provided to system 10.

While not limiting nor exhaustive, the following table provides examples of IMDs 70 that provide information to system 10, directly or indirectly and/or are controlled via system 10.

Pacemaker/Implantable Pulse Generator (IPG)
Defibrillator
Implantable Cardioverter/Defibrillator (ICD) (Pacing and Defibrillation)
Implantable Drug Pump
Externally Worn Drug Pump
Neurological Stimulator (brain, nerve, spine)
Gastric Stimulator
Stomach Therapy Device - (nausea, hunger, obesity)
Gastrointestinal Therapy Device (Irritable Bowel Syndrome, constipation,
incontinence)
Urinary/Bowel Control Device
Artificial Pancreas
Artificial Kidney
Stent
Spinal Cage
Spinal Disk
Implantable loop recorder (pressure, rhythm)
Artificial Heart
Mechanical Heart Valve
Biologic Delivery Device
Ocular Implant
Insulin Pump
Chemotherapy Delivery Device
Radiation Delivery Device
Neuro-Cardiac Stimulator (vagal stimulation)
MEMS/Nanoscale Actuators (clot removal/destruction/retention)
Cardiac Potentiation Therapy (CPT) Device
Muscle Simulator (diaphragm, esophagus, skeletal)
Cochlear Implant
Cardiac Resynchronization Therapy (CRT) Device

In addition, FIG. 3A illustrates a number of implantable sensors 82, 84, and 86 (collectively sensors 80). These sensors may be independent and simply output data. Alternatively, such a sensor may provide data to the IMD 70 and as indicated above may monitor one or more parameters of any given IMD 70.

The following is a non-limiting, non-exhaustive list of implantable sensors that may provide data to system 10.

Temperature Sensor
Heart Rate Sensor
Cardiac Output Sensor
Cardiac Rhythm Sensor
Stroke Volume Sensor
Ejection Fraction Sensor
Pressure Sensor (Arterial, Venous, Endocardial,
Epicardial, Pericardial, Respiratory, Renal,
Gastrointestinal, tissue (e.g., aneurysm))
Implantable External Pressure Reference Sensor
Lung Wetness/Edema Sensor
Oxygen/Oxygen Saturation Sensor
Carbon Dioxide/Carbon Dioxide Saturation Sensor
Respiration Sensor
Transthoracic Impedance Sensor (respiration
and/or defibrillation, pacing threshold)
Minute Ventilation Sensor
C Reactive Protein Sensor
Protein Sensor
Elemental Sensor (N, Na, K, Cl, Ca, etc.)
pH Sensor
Lead Position Sensor
Lead Integrity Sensor
Cardiac Contractility Sensor
Vascular Dimensional Sensor
Vascular Integrity Sensor
Vascular Topographical Sensor
Cholesterol Sensor
Blood Chemistry Sensor
(T Cell, K, INR, glucose, creatine, BUN, BNP, ANP, troponin)
Salinity Sensor
Dimensional/Volume Sensor (Size, Shape, Position of Heart)
Rhythm Motion Sensor (Motion, Pattern, Modeling)
of Contractual Cardiac Motion Sensor
Surface EKG Sensor (Subcutaneous, Device Mounted (e.g., “can”))
EGM Sensor
EEG Sensor
Neural Activity Sensor
DNA Analyzer/Sequencer
RNA Analyzer/Sequencer
Fluid Viscosity Sensor
Fluid Flow Senor
Turbulence Sensor
Alcohol Sensor (blood, breath, cellular)
Insulin Sensor
Glucose Sensor
Lactate Sensor
Hormone Sensor
Human Chorionic Gonadotropin (hCG) Sensor (Pregnancy)
Fertility Sensor
Estrogen Sensor
Testosterone Sensor
Adrenaline Sensor
Drug Sensor (presence, concentration)
Nicotine Sensor
Position Sensor (relative body position)
Motion/movement/activity Sensor (single or multi-axis)
Location Sensor (GPS)
Optical Sensor (visual, infrared, X-ray)
Imaging Sensors
Acoustic Sensor (heart rate, heart rhythm,
respiration, flow, thickness, viscosity)
Doppler Sensor
Partial Pressure Sensor (oxygen, carbon dioxide)
Nitric oxide Sensor
Ultrasound Sensor
Ischemia Sensor
Spectral Analyzer
Nerve Conduction Sensor
Stroke, Stroke Indicator Sensor
Seizure Sensor
Enzyme Sensor
Serotonin Sensor
Cellular State Sensor (e.g., cancerous)
Cancer Marker Sensor
Noradrenaline Sensor
Dopamine Sensor
Neurotransmitter (or surrogate) Sensors
Neural Activity Sensor
Hemorrhage Sensor
Ambient/Environmental Sensor (light level,
humidity, temperature, elevation, air quality)
Magnetic Sensor
Hall Sensor

There are also a number of external sensors 92, 94, and 96 (collectively 90) that collect patient data or information. External sensors 90 may be an electronic device that communicates data directly to the system 10. Alternatively, external sensor 90 may provide data that is then manually input into system 10 by the patient 20 or an observer. For example, patient weight may be recorded on an electronic scale and the weight data is transmitted to the information manager 30. Alternatively, the patient obtains weight data and records that data into a format that is then transmitted to the information manager 30.

The following is a non-limiting and non-exhaustive list of exemplary sensors or sensed parameters that may be used externally. It should be noted that many of the above internal or implanted sensors have external versions whether repeated herein or not.

Scale (weight)
Height
Dimensional Measurements
BMI
Body Composition
DNA/RNA Sequencer
Blood Chemistry
Urinalysis
Blood Pressure
Glucose Sensor
Pulse Sense
Oximeter
Electronic Pill Box
Temperature (patient, ambient)
Biopsy
Microphone (voice data, respiration, heart rate/rhythm, snoring)
Patient Input (Quality of Life Data, symptoms, concerns, journal/log,
compliance)
Imaging (video, still, infrared, X-ray, MRI, CT, PET)
EEG Sensor
EKG Sensor
Patient Identification
Patient activity (smart home technology e.g., time in bed, chair,
amount of water, etc.)

In summary, one or more parameters are sensed with respect to patient 20. In addition, patient 20 has access to a realm of information 100 that is relevant to the system 10. This information includes both subjective and objective personal knowledge about the patient, the patient's condition, symptoms, pain, circumstances, compliance with prescribed therapy, etc. The information 100 would also include educational content derived from any number of sources by the patient 20 that would prove useful to a recipient on the system 10. For example, a knowledgeable and proactive patient 20 (or concerned family member, friend, companion, etc.) may research potential conditions based upon personal insight and offer an interpretation through the system 10. This may potentially include a self-diagnosis that would otherwise not be possible with only the automated sensors 68, 80, 90 available for a given patient 20. The information manager 30 evaluates that self-diagnosis and as a result, more information could be requested; sensing parameters enabled, disabled or added, or the information may simply be passed onto a recipient. The information 100 is representative of data that is not objectively sensed or gathered by a device, but that is otherwise provided to the system 10 via the patient 20. Information 100 may take various forms, such as electronic media, voice data/communication, printed material, or written material delivered via facsimile, scanned electronically, or manually delivered.

FIG. 3B is a schematic diagram illustrating one embodiment of a system for collecting information from the patient 20. The patient 20 may have one or more sensors 80 implanted. In this example, the sensors 80 are un-powered sensors; that is, lacking internal power supplies. Power is provided by the telemetry interface 110, in this embodiment a reader post 111, via radio frequency (RF) transmission or some suitable format. The reader post 111 is positioned in a convenient location in, e.g., the home of patient 20 in a free standing application or incorporated into another structure. For example, the reader post may be incorporated into or onto a doorframe or wall where the patient 20 passes by an a regular basis, such as near a restroom, bedroom or entryway. The reader post 111 may also be mobile and/or portable and of appropriate dimensions forrepositioning/relocation for convenience of the patient 20. Thus, each time the patient 20 passes by the reader post 111, power is delivered to the sensors 80 which then sense the appropriate parameters. Information is communicated to the reader post 111 and transmitted to the appliance 120. In one embodiment, the sensors 80 are RFID tags configured to sense a given parameter and transmit data.

Other sensors 90 may be physically positioned with the reader post 111, such as a scale 90. Thus, the patient 20 steps on the scale 90 and weight information is collected and transmitted to the appliance 120 (which could be incorporated into the reader post 111). During the same time period information is collected from the sensors 80. Again, this structure could be a stand-alone sensing platform positioned within a home or health care environment, such as a hospital, clinic or nursing home. Alternatively, this structure could also be incorporated into a preexisting structure. The reader post 111 would form a portion of a wall or doorframe and the scale 90 would be mounted in or onto the floor so that data is effortlessly collected from the patient 20.

While the reader post 111 provides power to sensors 80 in this embodiment, such a reading device could also be utilized to stimulate/interrogate and/or collect telemetered information from selfpowered or internally powered sensors or medical devices. In addition, in an environment where multiple patients are scanned, RFID tags or similar devices may contain information to be used touniquely identify the patient 20 and assure that the collected data is matched with the correct patient.

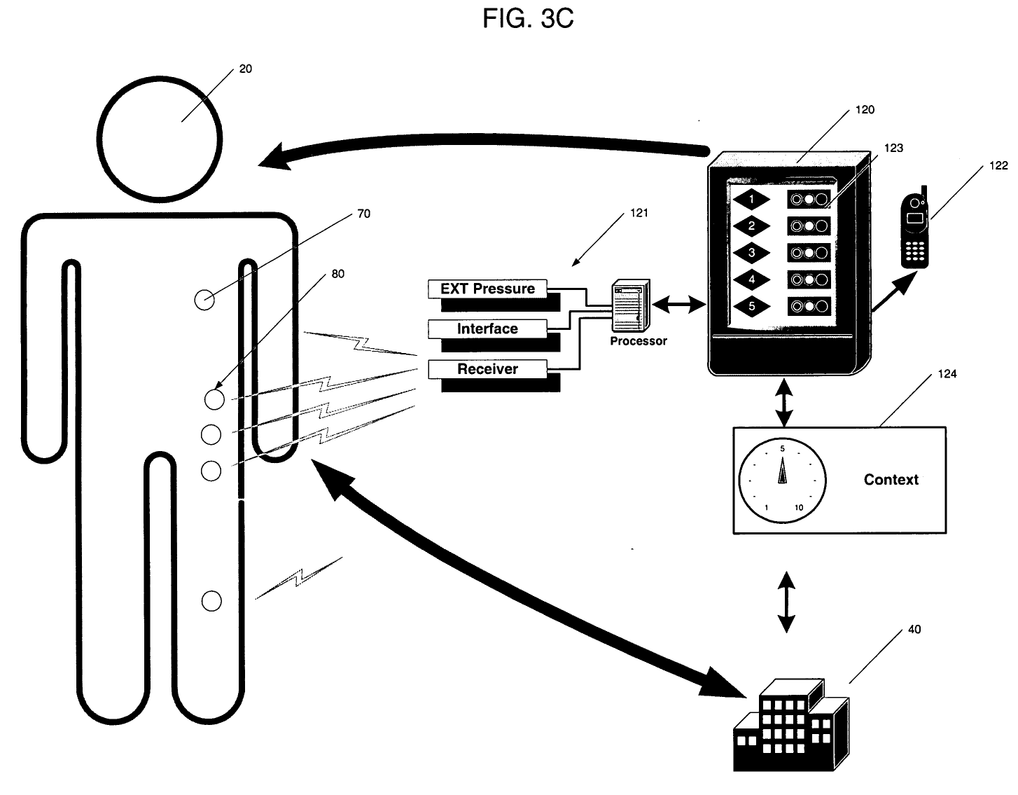

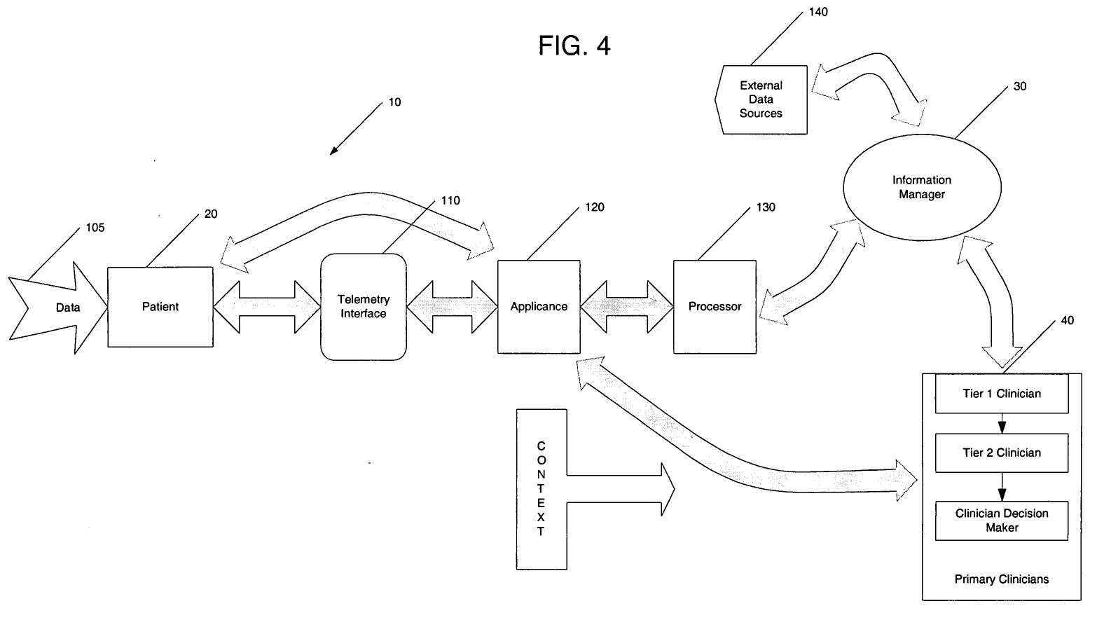

FIG. 4 is another schematic illustration of a subset of system 10, illustrated in FIG. 1. Data 105 represents the sensed or collected data from the various sensors 68, 80, 90 and/or the relevant information 100 that is provided to the system 10. As indicated, data 105 is directed towards information manager 30 and clinician 40, with context being defined or added in the process.

The clinician 40 may include a hierarchy of members, referred to as tier 1, tier 2, or the decision maker. This conceptually represents that multiple parties may review the information from the information manager 30 with varying degrees of decision-making authority. System 10 can route the data to the appropriate tier based upon factors such as criticality and urgency. That is, if life-threatening conditions exist, the system 10 will direct information to the decision maker. Additionally, in the course of patient care, the various tiers may take intermediate steps that lead to additional information requests or additional data sources that are incorporated by the system 10. For example, a nurse may evaluate the information and determine that the physician, the decision maker in this case, will require some additional information in order to make the decision. Thus, that additional information is requested prior to presenting the information to the physician. Failure to obtain an expected response from a given tier can result in automatically communicating with a different tier.

Of course, while not illustrated here, the patient 20 or an IMD 70 may use some or all of this data 105. For example, data indicative of ventricular fibrillation will cause an ICD to deliver a defibrillation therapy without input or response from the information manager 30 or clinician 40. Subsequently, this action will form a part of data 105. Similarly, weight data provided to the patient 20 via, e.g., an electronic scale and included in data 105 may alert that patient 20 to an undesirable weight gain or loss and trigger a change in behavior without input or response by information manager 30 or clinician 40. The patient's actions create new information that then becomes part of data 105.

In order to direct the data 105 from the patient 20 to the information manager 30, a telemetry interface 110, appliance 120, and processor 130 are provided within system 10. While illustrated as separate components, one or more may be combined together or include additional components that share or duplicate functionality.

Telemetry interface 110 is representative of the component(s) utilized to gather data electronically from IMDs 70, sensors 80, and/or sensors 90. For example, a given IMD 70 will typically include a power source, an antenna and a transceiver. Data is transmitted from the IMD 70 to an external receiver. Various telemetry platforms exist that define the range of such transmissions. For example, near or close range transmission typically requires a “programming head” or similar receiver to be placed adjacent the IMD via contact or close proximity to the patient's skin. The programming head is inductively coupled with the IMD 70 and information is transmitted in that manner. So-called distance telemetry allows the IMD to transmit over longer ranges such as three to fifty meters, or greater, typically as an RF transmission through media. In that case, the receiver may be located anywhere within the appropriate range and the IMD may transmit without required interaction by the patient. U.S. Pat. Nos. 6,240,317; 6,292,698; 6,009,350; and 5,324,315 illustrate various telemetry platforms and are herein incorporated by reference in their entirety.

Sensors 80 similarly communicate with the telemetry interface 110. Depending upon the specific sensor configuration, the same or similar telemetry mechanisms may be employed. Some sensors 80 may not include sufficient power supplies and/or transmission capabilities to transmit data in a manner similar to the IMD 70. In such cases, data may be communicated from the sensor 80 to the IMD 70 for subsequent transmission. Alternatively, power may be provided via an external source. For example, RF energy may be directed from an external source to the sensor 80. This may serve to charge a battery or capacitor permitting subsequent transmission. The sensor 80 may act as an RFID transponder, wherein the RF energy is again externally supplied and modified by sensor 80. Alternatively, the sensor 80 could be physically extracted and queried.

The external sensors 90 may include wired or wireless data transmission capabilities and transmit data to the telemetry interface 110. The telemetry interface 110 is not limited to a single data communication format, but may receive data from various sources in a variety of ways. For example, a given telemetry interface 110 may include a programming head (or similar structure) to interrogate an implantable device, a receiver or transceiver for receiving wireless communications in one or more frequency ranges, optical inputs, infrared inputs, or hard wired connections. This permits a single structure to receive inputs from a variety of devices. Alternatively, multiple telemetry interfaces 110 may be employed to collect data from different sources.

In one embodiment, the telemetry interface 110 is a home monitor having a programming head and/or an RF receiver to receive data via a wireless connection. The home monitor is coupled with a communication platform, either via a wireless or hardwired connection. The communication platform may be an existing telephone line, an Internet connection, satellite based communication or wireless data/voice platforms such as cellular, PCS, GSM, WiFi, or any WAP (wireless application protocol) or similar protocol.

In another embodiment, the telemetry interface 110 is a personal data assistant (PDA), personal computer, wireless telephone, or similar device that includes a receiver to receive data from the IMD 70 or sensors 80, 90 and subsequently re-transmit that data to the information manager 30

In summary, the telemetry interface 110 is a device or subset of a device that receives data from the IMDs 70 and/or sensors 80, 90. Generally, the telemetry interface will receive wireless communications from at least one source but may also include hardwired connections to particular devices, such as an external sensor 90.

It is generally contemplated that the telemetry interface 110 is proximate the patient during use; that is within the effective transmitting range of the IMD 70 or other transmitting device such as sensors 80 and/or 90. As these transmitting ranges become longer, the telemetry interface 110 will shift to more distant locations or become more integrated with the implantable devices and/or sensors 80 for longer distance transmission. For example, the IMD 70 may be WAP enabled and directly access a digital wireless network without any intermediary device. In this manner, data is sent directly from the source to the information manager 30 over an existing wireless communication network.

In other embodiments, structured or nodal networks provide the communication medium. For example, the IMD 70 or sensor 80 or other transmitting device utilizes Bluetooth or a similar communication platform and accesses any available communicating device. That communicating device then communicates with one or more subsequent devices until the data is passed to the destination. These communicating devices could be dedicated and fixed communication nodes such as a WiFi “hot spot” or random devices that the patient 20 passes in proximity with when communication is initiated. For example, a communications network could be employed that comprises a mesh architecture, a star architecture, or a hybrid star-mesh architecture. An appropriate scheme using ad-hoc and/or master/slave approaches may be used concurrently with peer topeer connectivity. Such random devices could include other patients with implanted devices having communication capabilities. In such instances, patient data must be properly protected and to the extent such data is relied upon for medical evaluation, proper error checking and time stamping would be provided.

The appliance 120 is a device that provides information to the patient 20. The appliance 120 and the telemetry interface 110 are commonly housed in many embodiments. For example, in one embodiment the appliance is a home monitor that includes the telemetry interface 110. In one embodiment, the home monitor includes lights or other visual indicators that indicate when a transmission from the IMD 70 or other transmitting device is in progress and when complete. Similarly, a visual indicator may be provided that indicates the information manager 30 has successfully received the data. Furthermore, in some embodiments, the appliance 120 includes multiple components. For example, the above described home monitor and a personal computer that is communicatively coupled with the information manager 30 via a private network (e.g., VPN) or a public network (e.g., Internet, world-wide-web) from the appliance 120. Thus, the monitor provides information regarding transmission status and the like and the personal computer is used to access more detailed information.

In other embodiments, the appliance 120 includes a stand-alone device, such as a home monitor, that provides more detailed levels of information. For example, a screen is provided that displays text or graphical information, a speaker may be provided to present audio information, a printer may be included to print information, and/or other information presentation mechanisms are provided. As such, the appliance 120 may be a stand-alone device or may be incorporated into any number of portable devices or generally stationary devices such as, without limitation, a personal computer, printer, cellular telephone, digital telephone, land line telephone, PDA, television, monitor, web enabled television/monitor, audio component, cable/satellite decoder (box), personal video recorder (PVR), modem (cable, wireless, telephone, etc.) or pager. The implanted device itself may include the ability to provide information to the patient 20 from data it has collected itself or received via communication with the information manager or other devices, and in that role, function as the appliance 120. For example, the implanted device may include a speaker or vibratory alert to present information to the patient.

FIG. 3C illustrates one embodiment of the appliance 120 that provides information and feedback to the patient 20. In this embodiment, the appliance 120 receives data from various sensors 80 and/or IMD 70. The appliance 120 includes incorporated components 121 such as a receiver and/or telemetry interface 110; a communication interface to couple the appliance 121 with another device (e.g., home computer); a processor; and may include its own sensors, such as the illustrated external pressure reference sensor or a glucose sensor. The appliance 120 is coupled with a wired or wireless communication network 122 to transmit information to the clinician 40. Feedback is provided to the patient via a display screen 123. In one embodiment, the status of various parameters is displayed to the patient. For example, the displayed information may indicate whether the patient 20 has or has not taken certain medications, whether blood pressure is elevated or normal, is whether glucose is within a desired parameter, and/or whether fluid pressure (edema) is elevated. In this manner, the system 10 can provide appropriate information to the patient 20 to facilitate self-care and avoid the need for medical intervention or a worsening of symptoms or conditions.

In summary, data is collected via sensors 68, 80, 90 about the patient 20 and/or provided by the patient. This data is directed to the information manager 30 via the telemetry interface 110 and the appliance 120 provides information to the patient 20. During this process, context is being defined and incorporated.

To this point, data transmission has generally flowed toward the information manager 30. It should be appreciated that the appliance 120 may communicate with the IMD 70 or the various sensors 68, 80, and 90 based upon the sensed data and/or patient input. For example, in one embodiment the appliance 120 may take the form of or include a patient activator. The information collected and presented to the patient 20 indicates that the patient 20 is having an atrial arrhythmia, specifically atrial fibrillation. One therapy option is an atrial defibrillation shock. The patient 20 may or may not wish to receive defibrillation and using the patient activator/appliance 120 can program the IMD 70 accordingly (within predefined parameters).

Similarly, the appliance 120 may include processing capabilities and/or algorithms beyond those available to the IMD 70 or sensors 68, 80, and 90. Thus, the appliance 120 may interpret the received data and initiate programming changes. For example, a given sensor 80 may include an array of sensors and over time one such sensor malfunctions. The appliance 120 determines that the sensor is malfunctioning based upon the collected data and disables the sensor from subsequent use.

Thus, data does not just flow towards the information manager 30, but intermediate devices or the patient 20 may intervene based upon that data and affect patient or device action.

Returning to FIG. 4, the appliance 120 is communicatively coupled with a processor 130 generally representative of the capability of the system 10 to interpret and act upon the data 105 and define context. Thus, the system 10 may include multiple processing devices and capabilities. Likewise, these processing capabilities may be distributed in one or more components throughout the system 10.

Typically, a cardiac IMD 70, such as an ICD, includes a sophisticated processor as well as software and algorithms to deliver therapy and respond appropriately to dynamic physiology. Thus, the IMD 70 forms a portion of the processor 130; the actions taken and relevant underlying data as well as the relevant data analysis results are provided to information manager 30 but the initial action taken based upon that data is based on processing that occurs in the IMD 70 itself. Since the ICD delivers potentially life saving and life sustaining therapy, the device necessarily includes certain internal processing capabilities; however, the greater the resources devoted to processing the greater the toll on the battery. As such, even an IMD 70 having a capable and robust internal processor might benefit from “outsourcing” non-critical but fully automated computational tasks to portions of the processor 130 outside of the IMD 70. The results and/or any programming changes are then communicated to the IMD 70, thus conserving battery power.

Similarly, as will be addressed in greater detail, an analysis of the data 105 and the generation of context may lead to a determination that additional information is required or that other steps should be taken. Such an analysis may be performed by the appliance 120 or by the information manager 30 depending upon the available resources. Thus, the portions of processor 130 external to the implanted device may be located within the appliance 120, the information manager 30, or even in remote locations (not illustrated). For example, the collected data 105 may be provided to an external expert system for analysis.

As the information manager 30 processes or reviews the processed data 105, context is defined for presentation to the clinician 40. In defining that context, the information manager 30 may determine that additional information is necessary or beneficial. Such information is gathered (if available) from external data source 140 and/or from the patient 20 and the various devices and sensors available.

With respect to the external data sources, the information manager 30 may access and extract information about the patient 20 from an electronic medical record (EMR), health information system (HIS), other medical records, other medical devices/instruments (e.g., data from a medical device programmer), insurance records, police records, emergency services records (e.g., ambulance reports, paramedic reports) or observers of the patient 20. Such observers could include friends, family members, neighbors, guardians, coworkers, and information from medical caregivers not available in the medical records. Such requests for information from observers would include email or other electronic messaging, automated voice requests, facsimile transmissions, mailed/delivered paper requests, and contact by human personnel associated with the information manager 30.

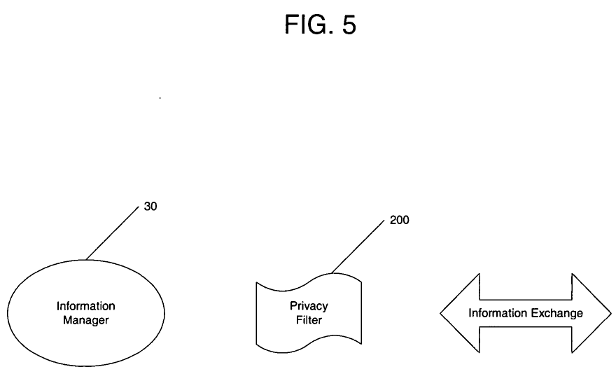

FIG. 5 is a schematic diagram illustrating that the information provided to and by the information manager 30 is often of a confidential and/or personal nature to the patient 20. In addition to simply respecting the patient 20, various rules and governance exist with respect to the treatment of patient records and data. For example, the Health Insurance Portability and Accountability Act (“HIPPA”) is Federal legislation that sets and enforces standards for confidentiality and security with respect to patient health data.

As such, a privacy filter 200 is included within information manager 30. Privacy filter 200 is meant to broadly illustrate compliance with any applicable rules, regulations and standards regarding the treatment of health information. Thus, information manager 30 will not contact a source (e.g., an observer) or provide information to any entity inappropriately.

Returning to FIG. 4, the information manager 30 may seek additional information from the patient 20. This could include enabling additional sensors already available or implanted, requesting that additional sensors are added, reconfiguring a given sensor (e.g., modifying thresholds or frequency of data collection), activating an electronde, or by requesting information directly from the patient 20. Again, such a direct request could be in the form of an email message, facsimile transmission, automated or human voice request (e.g., telephone call, voicemail), page, electronic communication with a dedicated device, or a mailed request. It may be necessary to provide instruction to the patient or caregiver. This could be done via video instruction on the TV, or computer appliance. The instruction could be to do something (e.g take your blood pressure) or provide how to do something (e.g. a video showing how to take blood pressure measurements). Medical information outside the scope of system 10 could also be requested. For example, the patient 20 could be directed to have laboratory testing done at a testing facility. If that facility is an external data source 140, the results are directed to the information manager 30 (manually or electronically communicated). If that facility is not an external data source 140, the results are provided to the patient 20 (or a caregiver) and the patient 20 then provides those results to the information manager 30. Failure to receive the requested information is an indication itself that can be communicated to the clinician 40.

The response provided then becomes part of data 105 that is considered and processed. Thus, the new information could lead to subsequent requests for additional information. The information manager 30 will evaluate the data 105 as completely and thoroughly as possible relevant to a given situation. To that end, requests for new or additional information from the patient 20 and/or the external data sources 140 would include any reasonably foreseeable and obtainable data while maintaining a reasonability and practicality standard. As such, there may always be the possibility of iterative information requests that build upon previously collected data.

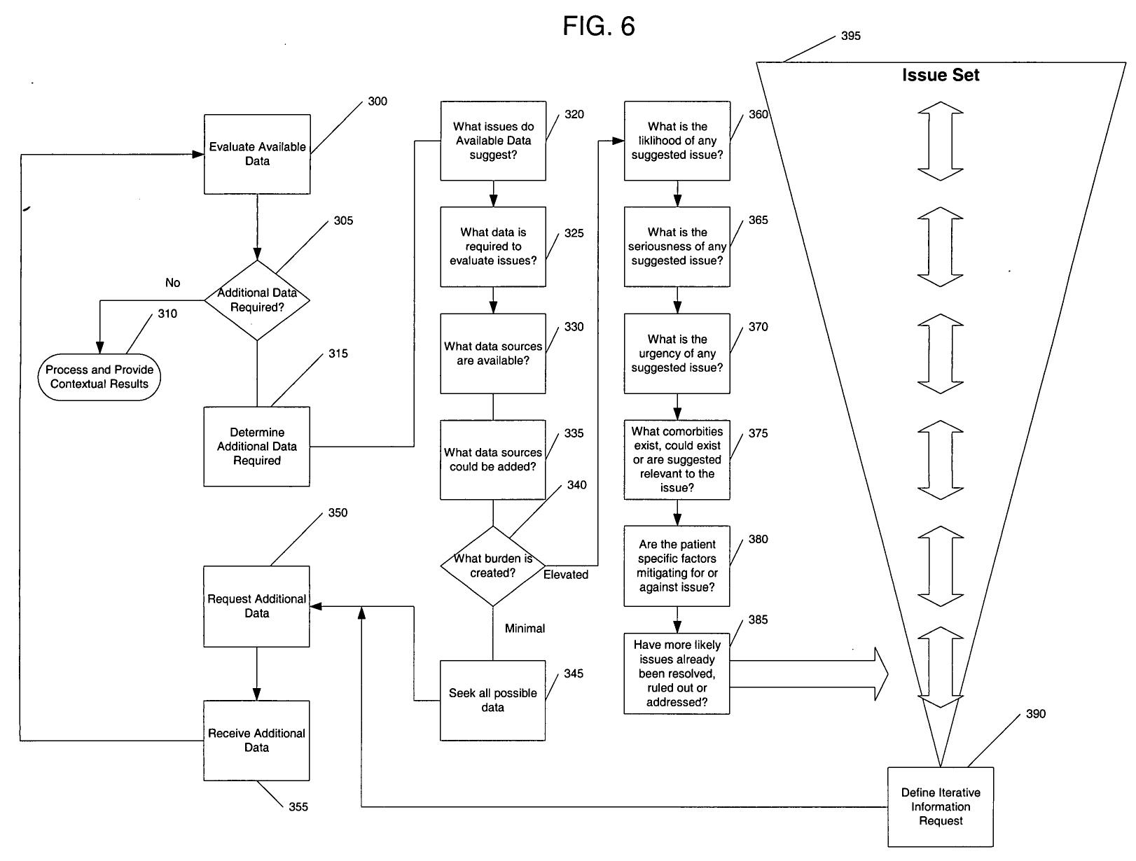

FIG. 6 is a flowchart illustrating a process by which the information manager 30 determines if additional information is required. At the point of evaluation, the available data is evaluated (300). This includes the data 105 made available at that point as well as records, information, and data previously stored with the information manager 30 either of a general nature or specific to the patient 20. The evaluation determines (305) whether any of the patient data indicates a need to gather additional information or that such additional information is necessary or beneficial to properly define context. If no additional information is required, then the information manager 30 proceeds (310) to process the information and provide the contextually relevant results to the intended recipient.

If the information manager determines that additional information is required, then the next step is to determine (315) what that information would include. When that additional data is identified, it is requested (350) in the appropriate manner. As previously indicated, this may include obtaining information from an external data source 140 or via communication with patient 20 and/or the IMDs 70 and appropriate sensors. Subsequently, this data is received (355) and the evaluation again occurs (300).

Returning to the step (315) of determining what information to request, an initial consideration is to identify (320) what “issues” are presented by the available data. As a practical matter, this step and those subsequently described are not necessarily sequential to steps 300-315, but are the basis for their outcome. Thus, identifying such issues is a part of evaluating the data (300) and determining if additional information is required (305).

The issue identified (320) may simply be an incomplete data set. For example, a communication error may have occurred and expected data is either missing, incomplete, or failed an error checking parameter. As such, this otherwise expected information would be requested. A more complex evaluation would include assessing the substance of the patient's data. For example, a patient having an ICD may begin to have nocturnal bradycardia, which did not previously occur. The ICD collects this information and also provides a pacing therapy to restore an appropriate heart rate. Thus, the symptom is addressed and both the symptom and the therapy are provided to the information manager 30 for subsequent reporting.

The “issue” would be what is causing the patient 20 to have the nocturnal bradycardia. While there are any number of causes and each would be evaluated in practice, this example will presuppose an outcome of the patient 20 having developed sleep apnea and the exemplary inquiry is directed as such and therefore should not be taken as in anyway limiting.

To continue, the information manager 30 has acquired the data indicative of nocturnal bradycardia episodes. In addition, the already acquired patient data indicates that this patient has gained a significant amount of weight. The patient 20 has recently provided self-assessments via email messaging to the information manager 30 indicating that he has felt fatigued as of late. Based upon this information, the issue becomes whether or not the patient may have sleep apnea.

The information manager 30 determines what additional data would determine whether the patient 20 does indeed have sleep apnea (325). Evidence of recurring cessation or severe inhibition of breathing for a prolonged period of time will indicate sleep apnea. The information manager then determines (330) what data sources could provide such information. Clinical determinations are often made in a controlled sleep lab with a sleep study involving polysomnography. Thus, having the patient enroll in such a study and having the results provided to the information manager 30 would resolve the issue. A respiration sensor that monitors patient breathing during sleep would also provide data indicative of sleep apnea. An oximeter would indicate oxygen desaturation that accompanies apnea and again indicate apnea. Other options are likewise available.

From this category of possibilities that would resolve or further clarify the issue, the information manager 30 determines (335) which, if any data sources are available to the system 10. In this example, the patient's ICD includes an impedance based minute ventilation sensor that is capable of sensing respiration and is not presently in use. Such information is provided in the patient record available to the information manager 30. There is no currently available oximeter or equivalent sensor and the other options are similarly not available for this patient without additional implantation. The controlled sleep study is an available option, but there is, in this example, no mechanism to facilitate the scheduling of such a study or the automated collection of the resulting data. It should be appreciated that system 10 would include such capabilities in various embodiments. In other words, the controlled sleep study is available but would require the patient 20 to enroll, participate and provide the results.

In summary, the information manager 30 identifies two potential mechanisms for acquiring the additional data. Activating an available respiration sensor is a first option and requesting the patient participate in a sleep study is a second option.

At step 340, the information manager determines what the burden would be for the various options. This determination has two considerations. The first, as implied by the example thus far, is that multiple options may exist for acquiring either the same or similar data or resolving the same issue. The individual options may be more or less burdensome to the patient 20, to the system 10, financially, or for other reasons. Thus, this is one aspect of the burden assessed at step (340)

The other burden aspect assessed at step (340) relates to acquiring data for multiple potential issues that may have been identified (320). As indicated, the current example presupposes sleep apnea as the relevant issues. Of course, bradycardia, fatigue and obesity (may or may not be related to on another) may relate to or indicate a vast number of other medical conditions from the quite plausible (e.g., drug interactions, heart failure, etc.) to the statistically unlikely (e.g., prolonged arsenic exposure). Multiple issues may relate to different causes or variations for a single condition as well as potentially relating to different conditions. For example, hypertension could relate to heart disease, heart failure, obesity, stress, or any number of “conditions”. In addition, if the patient has e.g., heart failure, a number of other variables specific to heart failure could be relevant to hypertension and thus define the areas of interest. In summary, identifying the issues to be considered forms part of the burden analysis of step 340.

Where the available information raises any issue, the information manager 30 would ideally acquire information to confirm or rule out all of those potential issues. Thus, the second burden consideration (340) is to determine the “reasonableness” of pursuing the various potential issues suggested or made possible by the data.

To the extent that the burden is low or minimal, all data sources are queried (345) to obtain information relevant to all of the issues identified. This information is acquired (350) and the process is continued. As an example, the data may suggest either a deficiency of substance X or a deficiency of substance Y is causing a particular condition. Statistically, a deficiency of substance X is far more likely; however, sensors are already available to measure for both substances. Thus, the likelihood of substance Y deficiency is low, but the burden is also low. Therefore, levels for both substances are sensed to have a positive determination rather than requiring sequential testing, which could prolong a more definitive resolution.

In determining the burden (340), a number of factors (360-385) are evaluated. These factors are correlated into an issue set 395 and the resolution of each factor will increase, decrease, or not affect a burden analysis of an iterative information request 390. The iterative information request 390 is the resolution of whether information manager 30 should investigate particular issues despite an elevated burden.

The factors are exemplary and are non-limiting. Furthermore, their order and individual importance or weight will depend upon the issues under consideration. One factor is the likelihood (360) of any given issue occurring. That is, the more likely a given issue, the greater the desire to investigate that issue despite an elevated burden in acquiring data. Conversely, if a given issue is very unlikely and the burden is elevated, a decision to pursue that issue becomes less likely.

Another factor considered is the relative seriousness (365) of the issue. The more serious or potentially serious the issue, the more likely the issue should be evaluated despite an elevated burden. For example, data indicating the possibility of occluded or partially occluded coronary arteries would likely be pursued regardless of burden and regardless of the number of potential alternative explanations for the data due to the fact that such occlusion is life threatening. Similarly, the urgency (370) of the issue is considered. Again, in this example, occluded arteries are likely to lead to sudden cardiac death (SCD) if untreated and once such arteries are occluded this not only may occur at any time, but becomes likely in the near term. Thus, this example is both extremely serious and urgent leading to a determination to pursue the issue regardless of burden. Of course, these are hypothetical analyses and the information manager 30 may recommend immediate medical intervention rather than or in addition to acquiring additional information.

Other bases exist for urgency determinations. For example, data indicative of infection such as HIV, SARS, or the like could be considered urgent in that the success of treatment is related to the promptness of beginning that treatment. Furthermore, simple awareness of the condition becomes urgent in that the patient may alter behavior to avert the subsequent spread of the disease.

It should be readily apparent how issues that are not considered serious and/or are not urgent would tend to decrease the likelihood of further investigation when an elevated burden is present. For example, information manager 30 would not likely require a patient to rush to a laboratory and have a tissue biopsy and analysis to confirm a suspected “dry skin” condition.

Similarly, seriousness and urgency may be used to determine whether or not the information manager 30 is the appropriate mechanism for pursuing the matter and may move the determination in either direction. Again, with reference to the issue potentially indicating occluded coronary arteries the extremely serious and urgent condition would likely (assuming the patient did not have an appropriate medical device or treatment option currently available) require immediate medical intervention and thus, the best course of action would be to facilitate that intervention rather than delaying the same to gather more information. Information manager 30 is productive in such situations in that the issue may be first raised based upon the data collected; the patient is directed to the appropriate resource (e.g., emergency room, calling 911, etc.); and data may still be collected and utilized in the course of that treatment. Of course, emergency response could also be directly contacted by the information manager 30.

Alternatively, even when issues are neither necessarily serious nor urgent, the unique capabilities of system 10 may provide the most efficient mechanism for resolving the issue, despite an elevated burden. Thus, seriousness and urgency are factors, but their use in determining subsequent action by system 10 is not one-dimensional.

Other factors considered include determining what comorbidities (375) may be relevant to the issue in question. This determination may interrelate with the analysis of likelihood, seriousness or urgency. For example, the patient may be known to have heart failure and the issue in question is whether sleep apnea is present. Sleep apnea is quite prevalent in heart failure patients, thus increasing the likelihood for this patient. Seriousness may be impacted due to the specific combination and/or likelihood of comorbidities. That is, the issue may often be associated with specific comorbidities that in a given patient may be more or less serious. Finally, the presence, absence and metrics of comorbidities may provide alternative routes to gathering information when direct determination of the issue is unduly burdensome.

At (380) the system 10 evaluates whether there are patient specific factors mitigating for or against a given issue. That is, what specific information is already known to information manager 30 about the patient that would make the issue more or less likely, more or less serious, more or less urgent, or relevant to other comorbidities. For example, if the issue is determining whether the patient has sleep apnea, the fact that the patient is obese would make sleep apnea appear more likely whereas a recent indication of bronchitis in the patient's EMR might suggest a more likely cause for current short term respiratory symptoms leading to current sleep issues.

Finally, as indicated, this process may be iterative (385). That is, the most likely issue or issues are addressed and if the result remains uncertain, then less likely and/or more burdensome inquires are then considered. The system could also take into account if previous tests have been performed, when they were performed, and look at what may have changed in the interim.

Once this analysis is complete, the issue set 395 is defined and formulated into the iterative information request 390. The system 10 then requests 350 the additional data from the appropriate source as previously indicated.

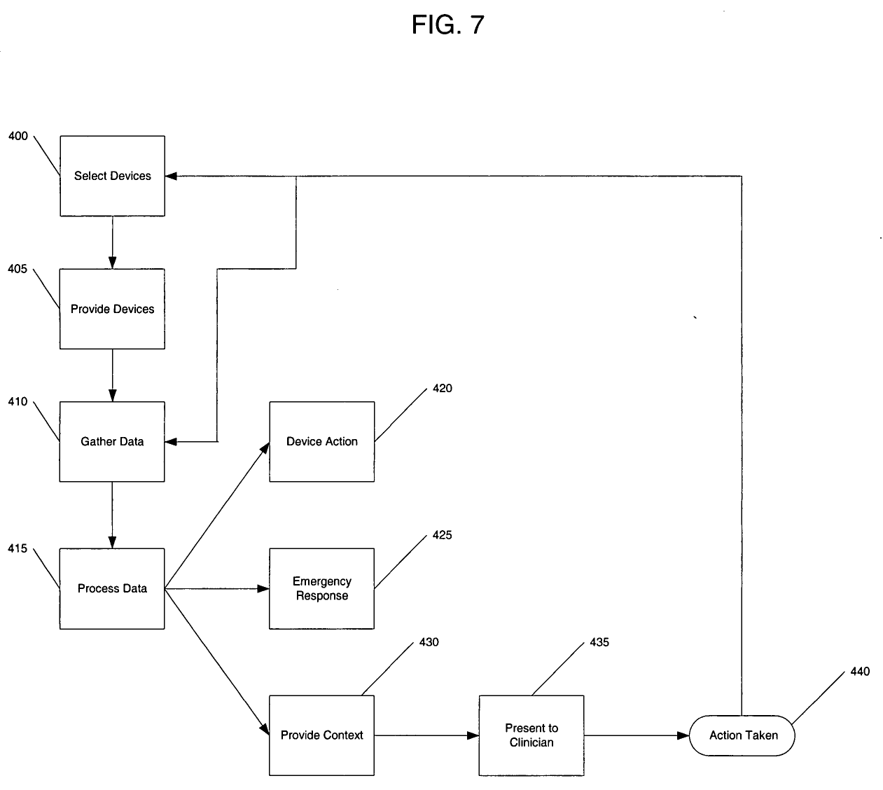

FIG. 7 is a flowchart illustrating a high level overview of a process for utilizing system 10. Initially, one or more devices are selected for a given patient 20 to provide information and/or connectivity to the system 10. Such devices could include the various internal or external sensors, internal or external medical devices, or communication devices discussed with reference to FIGS. 1 and 3. The selected devices are then provided (405) to the patient 20 or if already present, configured or programmed to appropriately interface with the system 10.

Over time, information is gathered (410) either in an automated fashion from one or more of the devices, through patient provided information, or from external sources.

This information is then processed (415). As previously indicated, this processing may occur in any number of locations including within an implantable device, within an appliance 120 proximate the patient 20 or by the information manager 30. Thus, the nature of the data determines the appropriate processing location.

In general, system 10 will undertake any of three actions based upon the processed data. One such response is to initiate (420) a device action. At a local level, a given IMD may process data and determine that the patient's heart is fibrillating and the device action is to deliver a pacing therapy and/or defibrillation shock. Similarly, an IMD may determine that glucose levels are elevated and the device action is to deliver insulin. The appliance 120 may receive and process data and determine that a given measurement or reference sensor is malfunctioning and instruct the device to recalibrate, disable that sensor, and/or contact the clinician. If available a redundant sensor may be activated or re-tasked. The information manager 30 may process data and determine that additional information is required and cause a device to collect data or adjust a parameter to facilitate such data collection. Such actions may, for example, include utilization of an internal or external noise sensor for noise cancellation, enabling or modifying a noise cancellation sensor, algorithm, or enabling or modifying a reference sensor/baseline reference device, algorithm or protocol. Similarly, the information manager 30 may enable or disable various therapy options as device actions.

Another response that may occur alone or in combination is to initiate (425) an emergency response. That is, emergency services 12 (FIG. 1) are contacted with respect to a given patient emergency. For example, a serious allergic reaction to a drug may incapacitate the patient 20 and the system 10 detects this condition. Emergency services 12 are contacted and an ambulance is directed to the patient's location. The device initiating the contact the emergency services could be an implantable device or the appliance that is coupled with an appropriate communication network. Alternatively, information manager 30 could generate such a request. It should be appreciated that one or more of these components may include a “panic” feature wherein the patient 20 can summon an emergency response via the system 10. For example, the appliance 120 may have a “panic” button or key. In addition, in certain situations the system 10 may contact emergency services based upon a lack of expected communication over a predetermined period of time. For example, if an elderly patient prone to falls fails to make a scheduled device interrogation session and has generally not missed such appointments in the past, then the system 10 may make attempts to communicate with the patient 20 and/or summon an emergency response. Such a feature should be used judiciously.

The third general action taken by the system 10 in response to the processed data would be to provide context 430. Again, this action may be taken alone or in combination with one or more of the previous actions. The information, in the proper context, is then provided to the clinician (435) and the clinician takes the appropriate action (440.

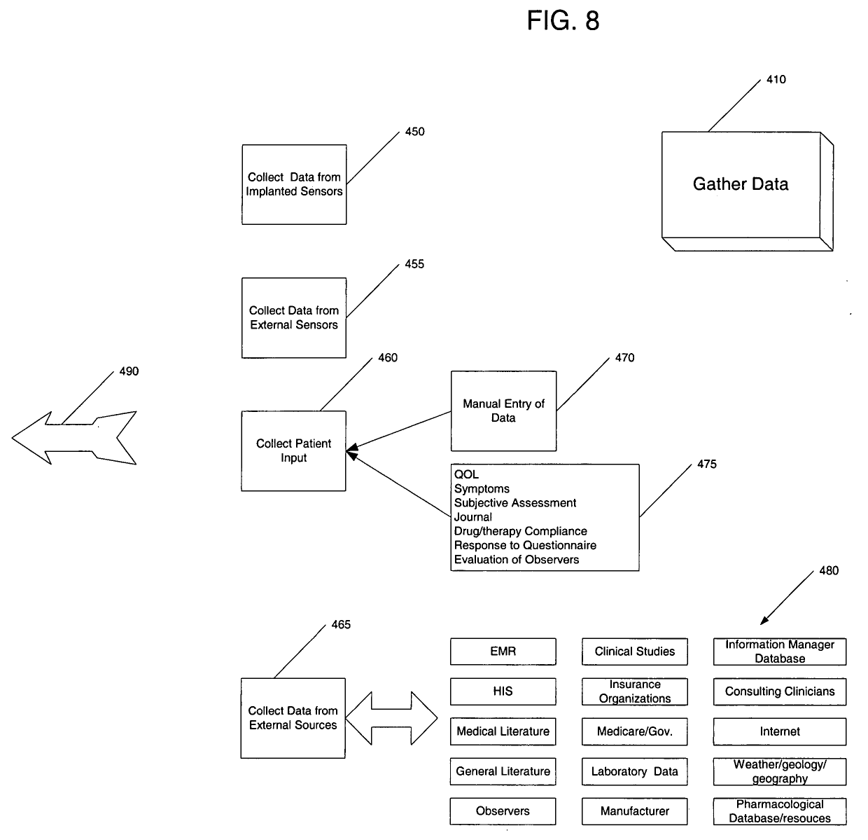

FIG. 8 is a block diagram schematically illustrating exemplary mechanisms for the step of gathering data (410). Data is collected from one or more implanted sensors 450 or external sensors 455. Such sensors would include, for example, those identified with respect to FIGS. 1 and 3. These sensor inputs (450, 455) are automated in that data is collected or transmitted without necessitating patient action or involvement with respect to the data itself (though patient action may or may not be required to engage the sensor). For example, external sensors such as blood pressure monitors or scales or implanted cardiac sensors collect data that is transmitted from the collecting device to the system 10.

Where patient involvement with the data is included, system 10 will collect patient input 460. This manual input 470 of sensor data could include patient readings of, e.g., a scale or blood pressure monitor. Additionally, the patient will provide other types of data 475 that is collected as patient input 460. Such information could include quality of life assessments, either subjectively described or in response to questions posed via the system 10. Other information may include the patient's description of symptoms, a subjective assessment of their current condition, journal entries, an assessment of drug or therapy compliance, or responses to various questionnaires offered via the system 10. In addition, observers (e.g., family, friends, etc.) may provide similar input or reporting to the system 10. Though indicated as collected patient input 460 and gathered in the same or similar way, the observer data may be isolated from the patient wherever appropriate or necessary. That is, while patient privacy concerns would require appropriate consent to gather data from a given observer, circumstances may warrant collecting that information directly without the patient's review of that data. This would tend to facilitate a more accurate and objective assessment.

This type of patient input 460 may be collected and input into the system 10 in a number of ways. For example, messages may be electronically communicated via e-mail, text messaging, paging, facsimile transmissions, document transmission (i.e. electronic document attachments), script response (e.g., Web forms), or the like using, personal computers, PDA's or similar handheld electronic communication devices or similar means. Various audiovisual communication formats may also be employed including telephonic communication with a live operator, voice messaging, interactive voice response, video transmission and the like.

Video transmission may relate to “sensor” data in that the visual representation is in and of itself useful for analysis. Alternatively, or in addition thereto, videoconferencing or the like may be used to facilitate the collection of the patient input 460. The collection of the patient's input via voice and/or video provides another metric for evaluation in that the information manager or clinician can identify factors about the patient's status. For example, elevated stress levels or incoherency may be readily apparent from a voice recording. Similarly, fatigue, sweating, shaking, discoloration, rashes, scarring, wounds and other physical parameters would be visible via video data even if not otherwise sensed or reported by the patient.

Gathering data 410 would include querying external sources 480 and extracting data. Examples of such external sources are illustrated and are non-limiting. Such sources might include patient electronic medical records (EMR). Data relating to a specific patient, similar patient groupings, medical conditions, medical practices or the like could be obtained from a health information system (HIS). Such a system might include the patient's EMR, but would also include a larger database of information. For example, the HIS may have profiles for given conditions or treatment pathways. The general medical literature, clinical study results/databases, the Internet/World Wide Web, pharmacy resources/databases may be queried. Related organizations such as insurance groups, HMOs, Medicare/Medicaid may be queried for patient specific information or for profile information. Laboratory data may be accessible in an electronic form or queries may be generated with responses returned in a suitable medium.

Another data source would be the manufacturer of the various medical devices or pharmaceutical, including drug interaction databases for these or government institutions. These institutions may provide extensive knowledge regarding their devices, operation, performance and patient responses.

The information manager 30 would generally maintain a database that may be queried. While not an “external” source, this database may be linked to such external sources to assure current or updated information.

The external sources 480 are meant to provide patient specific information and more general medical information. In addition, such external sources 480 could provide information beyond medical data. For example, external sources 480 could include weather, geology, geography, general knowledge and news related sources that provide information about the patient's physical environment and surrounding activities. The system 10 would be aware of the patient's location via self reporting or through tracking by utilizing a GPS sensor or even monitoring the source of data transmissions. This information would include, e.g., ambient temperature, humidity, elevation, air quality, and other factors that could affect the patient or impact various sensed data. News sources can correlate sensed data to real-world events that might impact patient health. For example, indications that the patient is proximate a hazardous site (e.g., chemical spill, major catastrophe, fire, war, earthquake, etc.) would provide context for various symptoms. In addition, this information could affect the burden analysis discussed with respect to FIG. 6. That is, what may be considered a relatively unlikely issue in general, e.g., arsenic exposure, becomes a much more likely issue when news accounts extracted from the external sources indicate high levels of arsenic present in the patient's location.

The data 490 collected from the external sources is provided to the information manager 30. Data from any given source could be obtained contemporaneously or sequentially. Of course, once data is gathered, the analysis of that data may reveal a need for additional data and lead to acquisition from another source.

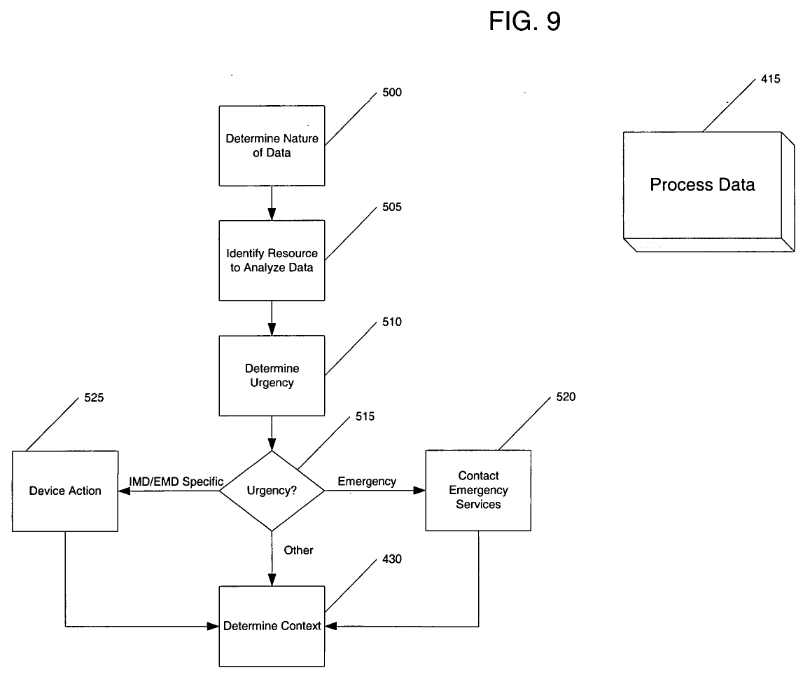

FIG. 9 is a flowchart illustrating in more detail the step of processing (415) the data. These actions may be performed at various locations throughout the system 10, including within an implanted device, within an external device, within an appliance or by the information manager. In addition, multiple processing components in one or more of these locations may act together to conduct any given data processing task.

Initially, the system 10 will determine (500) the nature of the data collected and then identify (505) the proper resource to analyze that data. In some cases this may be predefined. For example, an ICD will receive data indicative of cardiac rhythm. The ICD itself will process this data and respond accordingly. In other instances, a determination is made based upon the data itself. For example, the patient may input a wide variety of data via the appliance 120. Such data may relate to an acute situation that is best processed within the appliance 120 or the implanted device. The information manager 30 may best process other types of data and the data is routed accordingly. The system 10 will also attempt to quantify the quality, reliability and age of the data to act accordingly. For example, a patient reporting congestion occurred three weeks ago should be noted, but might not require any current consideration absent other variables.

The resource tasked with analyzing the data determines (510) the urgency of a given situation represented by the data. Urgency in this context is indicative of acute conditions that require an immediate medical response. If at step (515), the data indicates an urgent matter emergency services are contacted (520) and/or implanted or external device action (e.g., ICD defibrillation therapy) is taken (525). Included within device action (525), would be instructions to the patient to take some measure if appropriate. After the appropriate response is taken with respect to the urgent condition, the data is further processed, placed into context (430) and provided to the information manager 30. That is, while an acute response is necessary, subsequent reporting and analysis of that same data will be useful to a caregiver.

Similarly, when the data is not determined (515) to be urgent, the context is defined (430) and provided to the information manager 30. Many forms of data will simply be gathered and processed in this manner with no immediate action taken. This is not meant to exclude local data analysis that results in non-urgent device action, as this data is also placed in context and provided to the information manager 30 along this pathway.

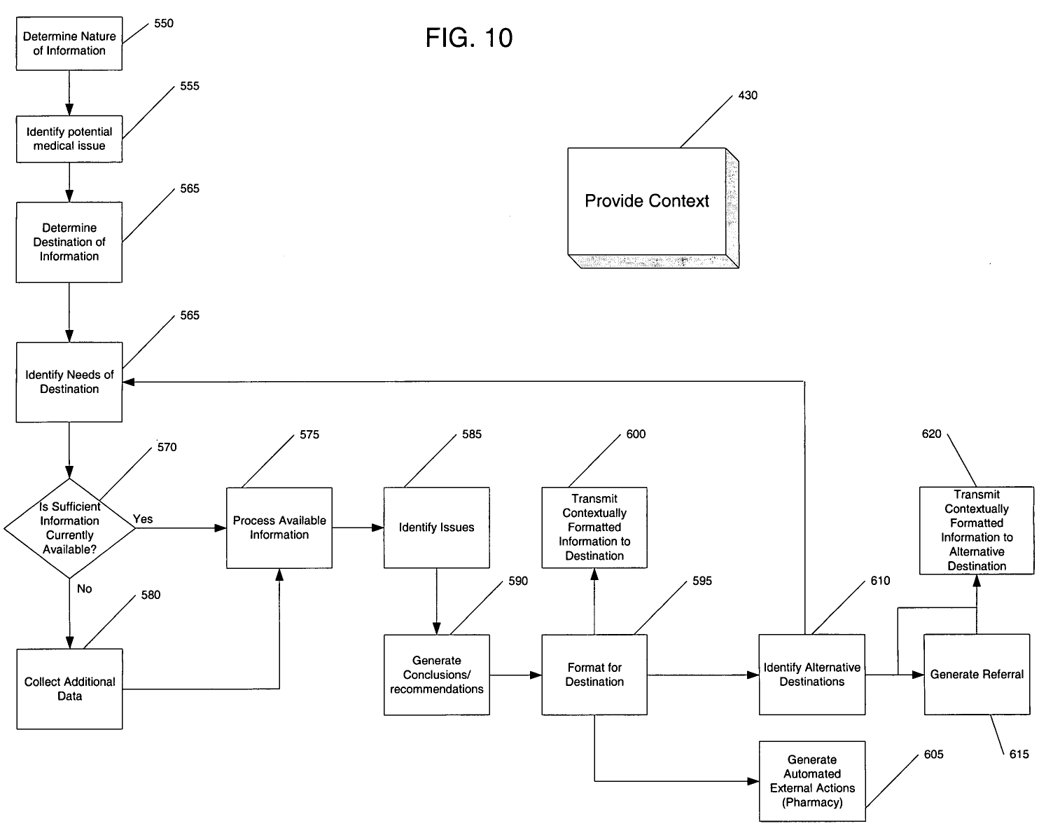

FIG. 10 is a flowchart illustrating a process for providing context (430). Initially, the data is analyzed and the nature of that data is determined (550) with respect to the particular patient. This would include a basic correlation of the data to its source and an identification of the type of data received to determine what rules or algorithms to apply.

Once known, the system 10 will analyze the data to determine (555) what, if any potential medical issues may be indicated by this data. This broadly includes any issue relevant to a caregiver, patient or other user of the system from, e.g., patient health issues to device performance and operation issues.

At this point, the system 10 determines (560) who the end user of the information, at least categorically, will be. That is, an indication of the primary clinician, secondary or alternative clinician 42 (FIG. 1), medical reviewer 44 (e.g., second opinion, insurance organization, governmental oversight), patient 20, or other user. For example, patients having an ICD will frequently transmit data related to the performance of the implanted device. Such data might include battery life, lead integrity, and similar parameters. The system 10 will determine that the cardiologist or similar caregiver is the intended recipient. The same device may provide other types of information such as heart rate, arrhythmia data, and therapy deliver history. This data would also be relevant to the cardiologist but may also be relevant to a general practitioner, internist, or other type of caregiver. Thus, steps (555) and (560) relate to identifying all the potentially interested parties and determining based upon the data, which parties should actually receive the data.

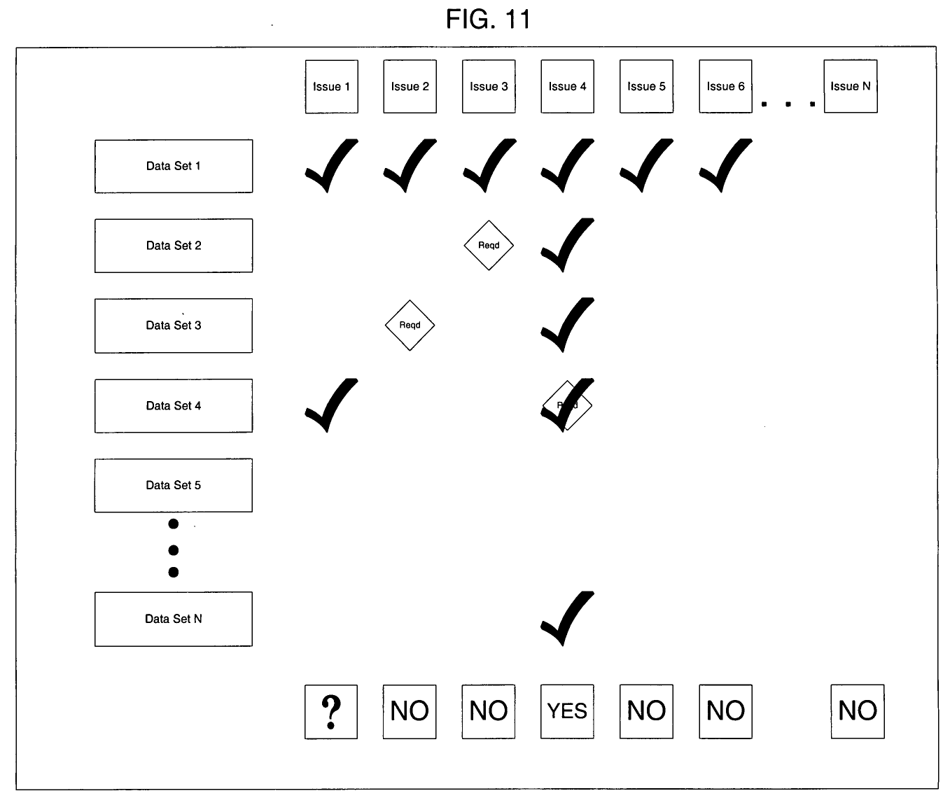

The specific approach taken to identify the appropriate destination will depend upon the data sources available to the system 10. In one embodiment, a database is provided that correlates available data to potential issues and directs data accordingly. FIG. 11 illustrates a schematic representation of one such database. Data sets 1-N represent the various sensory or informational inputs available to the system 10. These data sets are populated with the most current information provided from the sensors, obtained from the patient, or obtained from external sources. Though not limited as such, the data sets may be thought of as “symptoms” and the issues 1-N may be thought of as “diseases” or “comorbidities”. Thus, a given data set may represent a general concept such as whether or not the patient is obese (binary determination); a categorized concept (underweight, normal, overweight, obese); or specific concept (weight=X pounds). Multiple factors may be linked to define one or more of the concepts, such as specific height and weight data indicating whether the patient is overweight and/or to what extent. Similarly the issues may be general disease states (cardiovascular disease) or more specific disease states (sleep apnea) with the degree of specificity limited only by the particular data sources available.

Such examples are illustrative of the “symptom” and “disease” correlation. The data sets could also be more specialized and relate to particular device parameters (e.g., lead integrity, threshold settings, remaining drug capacity, etc.) and the issues would then relate to the device operation rather than diseases. For example, such device issues might include remaining battery life, device failure, lead failure and the like. Such analysis and classification of the data therefore serves two purposes; first it will facilitate the identification of potential recipients for the data and second it facilitates the presentation of that information in the most meaningful manner to that recipient.

Returning to FIG. 11, the exemplary database includes data sets 1-N and issues 1-N. Considering this example in the most general symptom and disease state model, the data sets are symptoms and the issues are potential diseases. A check mark indicates that the particular data set is an indicator for the disease, condition, or concern represented by the issue set. Each issue set will have a predetermined criteria for determining whether the system 10 will identify the issue set as relevant for subsequent dissemination.

Data set 1 might indicate a symptom that is generic to all or many of the issues, such as a patient report of a headache. Issue one could represent hypertension. Thus, a headache is a symptom consistent with hypertension and is so indicated. Data set 4 could be blood pressure data collected from an internal or external sensor. If that data indicates hypertension, then the system 10 would present issue set 1 to a caregiver in the appropriate context. Alternatively, data set 4 might represent the patient's weight or weight status. Thus, an overweight patient having headaches is also consistent with hypertension, but not dispositive. The system 10 could determine that the symptoms are too vague to consider; non-definitive and thus seek additional information as previously discussed; inconclusive but subsequent dissemination to a caregiver is warranted; or sufficient information is present to make the issue set likely and is presented to a caregiver as such.

While one may imagine a wide variety of symptoms that could be associated with any given disease or condition, some of those conditions may have “required” data sets that must be present to be indicatives of the issue. Some required data sets may simply be classification based. That is, to indicate and differentiate between heart failure classes (e.g., New York Heart Association Classification), data indicative of exertion capability (e.g., external sensor coupled with a treadmill), respiration at specific levels, or ejection fractions within certain ranges may be required for the system 10 to make a given classification. As another example, if reliable blood pressure data is available to the system 10, elevated blood pressure measurements might be required, regardless of other symptoms, before system 10 indicates a likelihood of hypertension.

Generally, the system 10 would recognize the potential for error in such an example and take into account that required conditions might be present even if not indicated by the sensor data. Thus, further inquiry may result if the symptoms are not adequately accounted for during the overall process or if the severity of a given issue set requires an abundance of caution. For example, in one embodiment the system 10 allows the ability to turn off a diagnostic or the display of certain data if a physician has looked at it and ruled it out, unless the evidence is strong and/or new information is available. However, the original information could be passed onto a new clinician. The system should allow for notes to be generated by the clinician as to why they are dismissing the data being presented. Likewise, the physician may want to just shut off an aspect of the algorithms because they have found it not very useful.

As indicated, issue set 4 has one required data set that is satisfied, along with a plurality of other present symptoms. Thus, system 10 determines that issue set 4 is indicated by the data and presented as such to the caregiver. System 10 identifies the caregiver that issue set 4 is most relevant to, and provides the data in the appropriate context.

With reference to FIG. 10, the appropriate destination (i.e., recipient) for delivery of information obtained and processed by the system 10 has been determined (560). The system 10 then determines (565) what information and what manner of presentation is most appropriate based on the needs of the intended recipient.

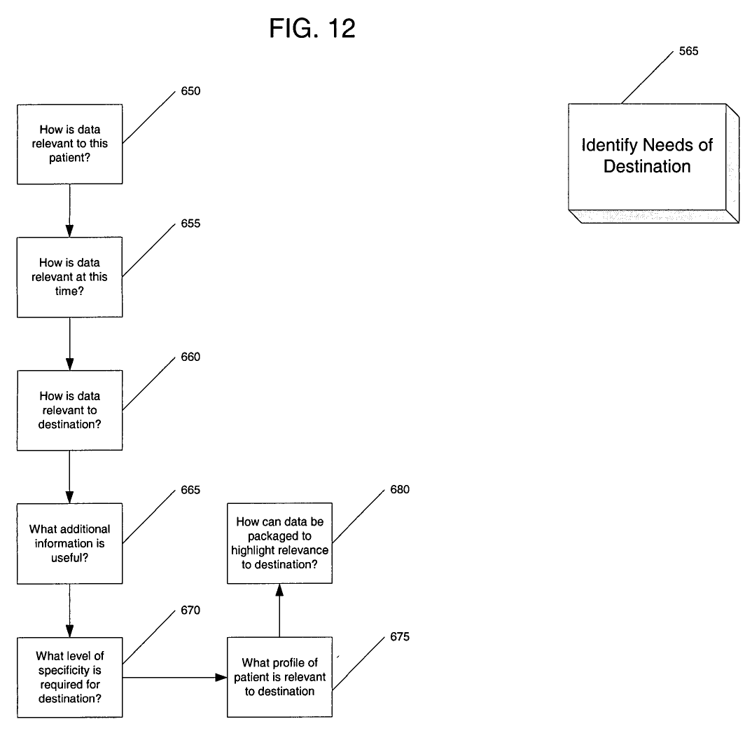

FIG. 12 is a flowchart illustrating one process for identifying the needs of a given recipient. The data or issues in question are analyzed to determine their relevance to the patient (650). As previously discussed, any given data point, symptom, or set of information may be relevant to a large number of issues. For example, weight data may be relevant to an issue of hypertension; thus, such weight data is being presented because system 10 determined that hypertension is a potential issue for this patient.

Similarly, the temporal relevance of the data is considered (655). The system 10 has identified hypertension as a current, unresolved potential issue thus making it relevant to a caregiver at this time. Weight data might not be relevant in this context subsequent to the resolution of the issue of hypertension. As another example, respiratory data indicating breathing difficulty might appear temporally irrelevant for a patient whose bronchitis has been long resolved, but would be temporally relevant to a current consideration of sleep apnea or asthma. Thus, temporal relevance is the correlation of given data to the issue or issues presented for consideration by the caregiver at the current time. The caregiver is then provided with a good understanding of what this particular data may be relevant to at the present time.

Thus, the relevance of the data to the patient and the patient's present circumstances are identified. In addition, system 10 determines (660) how that information is then relevant to the recipient caregiver. For example, a general practitioner (GP) receiving data indicative of hypertension would likely wish to treat the hypertension and would want appropriate accompanying information readily available. A heart failure specialist would also consider data indicative of hypertension relevant to the treatment or therapies already in place or that should be used in addressing the patient's heart failure. Thus, in this example, the relevance to the general practitioner is the treatment of hypertension while the relevance to the heart failure specialist is the indication that hypertension would have on the heart failure condition.

As indicated, the system 10 will identify (665) other information to the caregiver based upon the determined relevance. In the above example, the data is relevant to the GP in the context of treating hypertension. Numerous treatment options exist from dietary and lifestyle indications to the use of one or more drugs, for example. As such, the GP would be interested in whether any previous treatment has been attempted, the patient's compliance, the patient's dietary activities, the patient's activity level, the patient's current medication regime, drug allergies, and any risk indicators for drugs in general and in particular the drugs most likely used in the treatment of hypertension.

Much of the additional information identified may be drawn from expert systems, medical databases, practice guidelines or other available source. That is, for a given situation with a given type of caregiver, the system 10 will determine what additional information would most likely be useful to that caregiver. At the next level, the system 10 can move beyond general guidelines and best (or suggested) practices and include patient specific analysis as to what information is useful. Additionally, the system 10 will monitor the patients, the caregivers and their interactions and learn what resources, beyond those provided, are being requested by caregivers and in what circumstances. Thus, in future cases this learned architecture is combined with the best practices and patient analysis.

The system 10 must balance the availability of almost unlimited information with the ability of the recipient to make efficient use of that information. Therefore, in addition to determining what information to provide, as discussed above, system 10 determines (670) at what level of specificity to present the selected information.

Continuing with the relatively simple example of hypertension, the system 10 may have many data points representing blood pressure measurements made over time. The caregiver may only need or want to know that at some point over a relevant period, blood pressure exceeded some threshold or that the most recent measurement is too high or too low. Alternatively, detailed charts, graphs or tables could be presented with numerous measurements made over time. Averaged or other derived values can be extracted from the data and presented as an indicator. Additional external data such as weight, sodium levels, dietary intake, etc. could be correlated and presented for each data point on such a presentation. For a simple, routine follow up to confirm whether a prescribed medication is controlling hypertension the caregiver may require less detail; where identifying the cause of the hypertension is at issue, more detailed information could be provided. It should be appreciated that this process relates to the information and the format of the information presented to the caregiver in context; additional requests may be subsequently made by the caregiver from the system 10 to get other information or more detailed information.

Similarly, the system 10 determines (675) which information related to the patient profile is most relevant to the caregiver in the context of the issues being presented. In some cases, data from multiple patients may be batched together and presented for a review by a caregiver. For example, patients having an implanted device may have routine follow-up appointments. The follow-up appointments will usually indicate that there are no problems and the device is operating correctly. As such, a minimal patient profile is required, such as the patients name or even just a numerical identifier along with confirmation that the remote follow up occurred and the lack of issues.

When presenting unresolved issues to a caregiver, more detail is likely warranted. Types of information that might be included are the patient's name, any recent activity between the caregiver and this patient, or factors that may allow the caregiver to recall the patient; that is to trigger any memory or recollection the caregiver may have of the actual patient from the past. Other contextual information might include the patient's tolerance of new therapies, their past compliance, personal competence, what support network they have, their living environment or similar factors. Again, the system 10 identifies which, if any, of these types of information would best define the context for the selected caregiver.

Once the information to be delivered is identified, the system 10 determines (680) the most appropriate presentation of the information to highlight the relevance and the context to the specific caregiver. Critical information can be highlighted, bolded, colored or otherwise emphasized, but such issues are secondary for this step. Any given piece of information can be represented in numerous ways. The presentation selected can affect how the recipient perceives the information, the impact the information might have, and whether the recipient understands the information.

As an example, an implantable medical device may gather cardiac data. That data might indicate a patient is periodically having an arrhythmia, such as atrial fibrillation or atrial flutter. One way to present that information would be to provide ECG or EGM tracings of the cardiac data; essentially, this is raw data. A skilled EP or cardiologist may want this data and gather insight from the timing of the arrhythmias and the nature of the rhythm preceding the events. The system 10 could provide the “raw data” (e.g., the tracings) in an annotated form. That is, the portions indicating the arrhythmia may be marked with text or graphics; thus, a caregiver less adept at analyzing such tracings can still make use of the tracings and will be alerted to their significant features.

Rather than providing tracings, the system 10 could provide descriptive information such as “atrial fibrillation occurred at 1:15 a.m. and lasted for 5 minutes,” or “atrial fibrillation, five occurrences” or “patient has arrhythmias.” Alternatively, graphics or other identifiers could be utilized. In summary, the same types of information can be presented in various ways and in varying levels of detail that are tailored to the intended recipient. The nature of the presentation selected by the system 10 is therefore based upon the intended recipient.

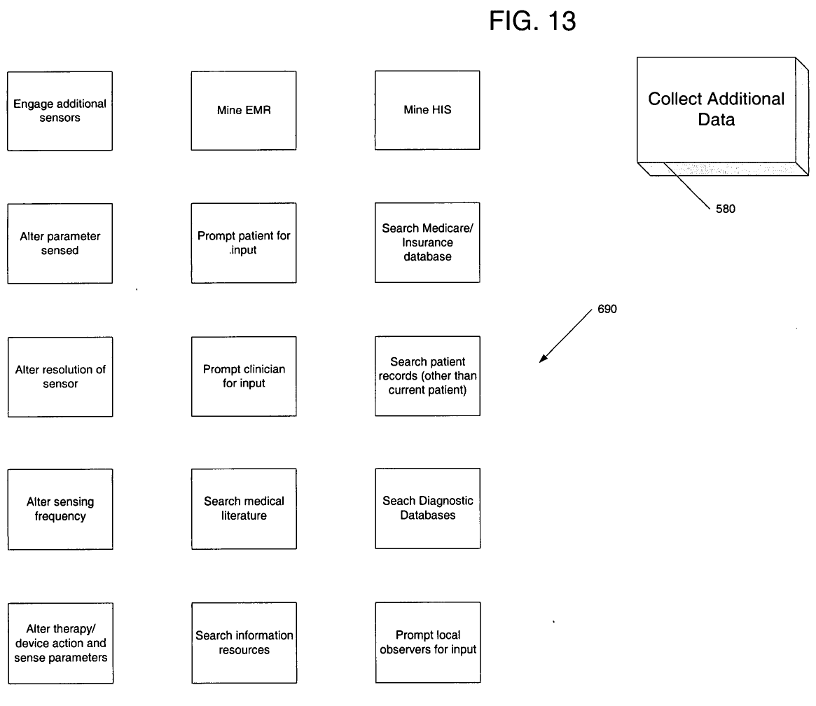

Returning to FIG. 10, the system 10 has identified the needs of the destination (565). Subsequently, a determination is made as to whether sufficient information is currently available (570). If not, the system 10 proceeds to gather additional data (580). This step has been previously discussed and FIG. 13 provides a non-limiting set of exemplary data sources (690). These data sources (690) include: additional sensors (newly added or present but previously inactive), altering what parameter is sensed, altering a resolution, frequency, or other sensor parameter, altering therapy/device and sensing results, mining an EMR, requesting information from the patient, requesting information from a clinician, medical literature, information resources (internet, etc.), health information system, Medicare/insurance database or resources, other patient records, diagnostic databases, and observers.

If sufficient information is available (570) or after additional information is collected (580), the system 10 processes all of the available data (575) and identifies (585) the issues. Of course, much of this has occurred in the previous steps, as discussed in detail.

To this point, system 10 has collected information, identified potential issues, identified to whom the information should be presented, and how that information should be presented. In many cases, the system 10 can take an additional step and generate (590) conclusions or recommendations. For example, in the hypertensive example, the data presented may indicate that the patient has hypertension and that particular dose of an ACE inhibitor is a recommended treatment. In addition, this particular drug is recommended because the patient is allergic to the alternative. A more general conclusion might be that the patient appears to be hypertensive; here is a list of the commonly prescribed medications, their side effects and contraindications. Again, the level of detail is selected by the system 10 specifically for the recipient.

With that, the system 10 has gathered and generated all of the information it will present to the caregiver at this point. This information is formatted as appropriate for the intended recipient (e.g., step 595) and provided in this contextually relevant format. The system 10 also includes a feedback feature whereby the actions of the caregiver are monitored and compared to any recommendation made by the system 10. Thus, the system 10 will effectively learn over time and is able to alter its various decision making algorithms based upon the results taking be a given caregiver for a given patient. Of course, the system 10 will recognize its own limitations with respect to information content. For example, a given set of facts may dictate a particular recommendation that leads to the patient being seen by a caregiver. During the course of that exam, the caregiver learns information that was not made available to the system 10 (or that a condition has changed). In such a case, the system 10 was not technically incorrect. If the caregiver fails to report this new information to the system 10, it would be improper for the system 10 to assume an error in its algorithms based solely on an unexpected treatment. Thus, the feedback loop would need to be complete to be most useful. Additionally, the system 10 can incorporate queries to obtain the information that was missing (but provided to the caregiver) to further increase effectiveness.

As discussed herein, the system 10 provides context for information. In addition, the recipient of such information can alter what is received to provide more or less detail and/or to alter the content received. FIG. 3C illustrates a content filter 124 that is controlled by the recipient, in this case, the clinician 40. In one version, the recipient can adjust the filter 124 to indicate the desired level of detail for the information presented. Of course, when an issue arises, the recipient can always gain access to a fuller data set from the system 10 and when warranted, the system 10 may determine that more information than requested in necessary in a given situation and present that greater level of detail to the recipient despite the filter selection.

The deliverable can be in any of a number of formats including printed matter that is physically delivered or sent via facsimile, electronic documents or messaging, other electronic data formats, voice transmission via a live operator or via automated generation.

It should be apparent that electronic transmission of data includes a wide number of approaches from email or paging to attaching electronic documents to a transmission. In addition, formatting for the destination (595) would also include preparing the information for a particular display device or format. That is, a given caregiver may receive data from system 10 via a wireless connection to a PDA. Thus, the data is formatted to the limited screen size. Similarly, various caregivers may utilize particular communication, billing, records, or other management applications, software or proprietary systems. As such, the data is formatted in a compatible manner.

Depending upon the level of decision-making authority provided to the system 10 by the caregiver and/or the patient, certain actions (605) may be taken in response to the conclusions and recommendations (590) that were made. For example, the collected data and the subsequent processing by system 10 may lead to a determination that laboratory or other clinical testing is required or the patient's drug regiment should be modified. System 10 can generate an automated request to the patient's pharmacy to fill a prescription for a particular dosage or even to add a new medication. Again, with proper authorization this may be a completely automated event; that is, the caregiver may have preauthorized that such action be taken.

Alternatively, without such authorization the system 10 may still submit the prescription to the pharmacy with the understanding that the caregiver will need to authorize the prescription prior to the pharmacy dispensing the drug to the patient. In this manner, the caregiver reviews all medication decisions, but to the extent the system's recommendation is approved, the prescription has already been prepared and this speeds delivery to the patient. Similarly, whether recommended by the system 10 or entirely by the caregiver, the caregiver can use the interconnectivity of the system 10 to submit prescriptions to the patient's selected pharmacy.

Through the above-described processes, data has been collected from various sources, processed, and placed into the relevant context for the intended recipient (e.g., primary clinician), and transmitted (600) to that intended recipient. The collected data may also be relevant to one or more alternative clinicians 42. For example, a heart failure patient may have multiple sensors that provide information. The system 10 reports a series of events as they relate to decreased cardiac output to the heart failure specialist. In addition, respiratory sensors indicate multiple cessations of breathing during nighttime hours. Thus, the system 10 determines that sleep apnea is at issue and that a sleep specialist or similar caregiver should examine the patient. Thus, the data relevant to the sleep specialist is gathered (565), appropriate additional information is gathered, and the combination is placed in the proper context for the sleep specialist. This information is then transmitted (620) to the sleep specialist for review.

If the alternative clinician 42 is already treating the patient, system 10 provides the information to that clinician 42 as previously described. When the patient has not already been seen by this clinician 42, then certain insurance practices, legal practices or medical standards may require a referral. System 10 generates such a referral (615) as required prior to submitting the contextually formatted information to the alternative clinician 42. The processes implemented by system 10 in determining that an alternative clinician 42 should be consulted may be a sufficient basis for the referral. Alternatively, the recommendation can be made to the primary clinician 40 or the medical reviewer 44, either of which may then review and approve the referral.

Medical reviewer 44 (FIG. 1) could include the patient's insurance company and/or could include an independent medical practitioner tasked with having the authority to review the recommendations made by system 10 and to authorize a given referral. The medical reviewer 44 can serve another function by reviewing the actions taken by the primary clinician 40 and any alternative clinicians 42 as well as the recommendations made by system 10. In this role, there is some oversight in assuring that the patients are receiving proper medical treatment. System 10 may provide for automated billing for various clinicians and/or insurance submissions.

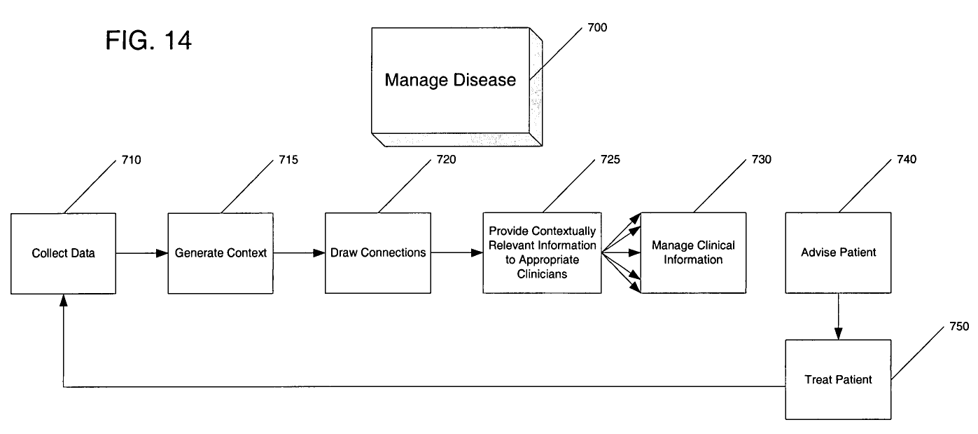

FIG. 14 is a flow chart illustrating a process by which system 10 is used to manage (700) a patient's disease state. Medical treatment, in general, is the presentation of a current set of conditions and the resulting diagnosis and treatment. For example, a patient arrives at a hospital with a broken arm. The physician likely obtains an X-ray, determines the bone is broken, treats any tissue damage, and sets and stabilizes the broken bone with, e.g., a cast. In a period of time, the patient returns and the physician removes the cast. Here, the physician has successfully treated the patient's condition.

If, prior to removing the cast, an infection developed the patient would return to the physician and the physician would reassess the situation. Perhaps antibiotics are sufficient; perhaps the cast needs to be removed, a wound treated and the bone reset. In either case, this patient is again receiving treatment for symptoms, as has been the accepted form of medical practice.

Chronic disease states, particularly those that involve multiple comorbidities are not as successfully resolved via the classic approach of assessing and treating current symptoms. Thus, disease management, as delivered via system 10, includes the collection, processing and distribution of patient state information and the presentation of that information in context to the proper caregiver at the most relevant time. In this manner, the patient's overall health is addressed and factors are managed on a continuing basis to maintain health rather than simply responding to symptoms that may occur or worsen before being evaluated.

As one example, heart failure patients suffer from a very serious condition but also are likely to have a whole host of comorbidities. The primary thrust of heart failure is the inability of the heart to deliver sufficient cardiac output. Thus, in a treatment approach a physician will take steps to maintain or increase cardiac output in response to the disease affecting the patient. In other words, when the symptoms worsen or become severe steps are taken to treat them.

In the disease management approach, system 10 monitors various data sources providing patient data. One such sensor 68, 80 is an edema sensor that determines fluid levels within the lungs. As fluid levels increase, system 10 denotes the change and through the processes described above recommends changes to the patient's treatment. This may then resolve or stabilize a situation prior to it degrading the patient's condition and requiring a more dramatic responsive therapy.

The system 10 is useful for disease management by collecting data (710) from a wide variety of sources, many of which would not be available to, relevant to, or manageable by any single caregiver. From all of these sources, system 10 processes the data and generates (715) context to assure the relevance of the information is provided and apparent to the appropriate caregiver. Since system 10 is not limited to a specific class or specialty of caregivers, data relevant to many disparate issues is acquired. The system 10 draws (720) connections from this data and provides (725) the contextually relevant information to an appropriate caregiver.

Thus, for any given patient multiple caregivers may be providing a variety of treatments, therapies and recommendations. The system 10 receives and manages (730) this data as well. Since these caregivers may very well be unaware of what the other has done, the system 10 incorporates all of this data and is subsequently used to identify issues and provide context. The patient, through this process, is advised (740) and treated (750). This treatment, the effects it has on the patient, the patient's compliance and similar factors continue to be monitored by system 10 and again, this data is incorporated and provided in context wherever appropriate.

We claim:

1. A patient management system comprising:
an implantable medical device having a sensor for sensing one or more patient parameters and a transmitter for transmitting data related to the one or more patient parameters;
an appliance external to the implantable medical device and having a receiver to receive the data transmitted from the implantable medical device and a communication interface to transmit information over a communication network; and
an information manager remote from the appliance and configured to receive the information transmitted from the appliance over the communication network, the information manager further including a processor and a context module configured to process the information received and generate context specific reporting information.

2. The patient management system of claim 1, further comprising:
a clinician interface accessible by a clinician and remote from the information manager to provide the context specific reporting information.

3. The patient management system of claim 1, wherein the implantable medical device is a cardiac device.

4. The patient management system of claim 1, further comprising an external medical device communicatively coupled with the appliance to provide external medical device data to the appliance.

5. The patient management system of claim 4, wherein the external medical device is selected from the group consisting of a: scale, blood pressure measurement device, and glucose measurement device.

6. The patient management system of claim 1, wherein the appliance further includes a patient interface.

7. The patient management system of claim 6, wherein the patient interface receives information input from a patient.

8. The patient management system of claim 6, wherein the patient interface provides information to a patient.

9. The patient management system of claim 8, wherein the information provided to the patient is generated by a processor within the applicance.

10. The patient management system of claim 8, wherein the information provided to the patient is generated by the information manager.

11. The patient management system of claim 8, wherein the information provided to the patient is generated by a clinician and provided via the information manager.

12. The patient management system of claim 1, wherein the information manager further comprises a recipient determination module that determines which recipient would find the information most relevant based upon the information received from the appliance, wherein the context module generates the context specific reporting information in a context chosen for the determined recipient.

13. The patient management system of claim 1, wherein the information manager further comprises medical issue identification module that identifies relevant medical issues within from the information received from the appliance.

14. The patient management system of claim 13, further comprising a supplemental information module that determines available additional data sources relevant to a resolution of the medical issue, communicates with one or more of the additional data sources and incorporates additional data into the context specific reporting information.

15. The patient management system of claim 24, wherein the implantable medical device is programmable and the appliance transmits programming instructions to the implantable medical device.

16. The patient management system of claim 15, wherein the programming instructions are generated within the information manager.

17. The patient management system of claim 16, wherein the programming instructions are generated in response to a communication from a clinician external to the information manager.

18. A method of managing a disease utilizing a patient management system comprising:
collecting physiological information from one or more sensors that sense physiological parameters for a given patient;
transferring the collected information to an information manager remote from the patient;
acquiring available patient data at the information manager;
evaluating the collected information and the acquired patient data and identifying medical issues;
determining if additional information is required to address the identified medical issue;
determining if the additional information is available;
determining whether to acquire the additonal information;
identifying a medical caregiver most relevant to address the medical issue; and
generating context relevant to the identified medical caregiver, formatting and selecting the medical issues and acquired information according to the generated context and making the contextually formatted and selected medical issues and acquired information available to the identified medical caregiver.

19. The method of claim 18, wherein determining if additional information is required to address the identified medical issue includes calculating a burden of acquiring the additional information.

20. The method of claim 19, wherein calculating the burden includes evaluating the likelihood of the issue, the severity of issue, the urgency of the medical issues, and the availability of sources to acquire the additional information.

21. A method of utilizing a patient management system for disease management comprising collecting and processing patient state information, determining from the patient state information a relevant caregiver, determining a context relevant to the caregiver, and presenting to the caregiver relevant processed medical information in the determined context.
